(19) United States
(12) Patent Application Publication
(10) Pub. No.: US 2006/0193832 A1
(43) Pub. Date: Aug. 31, 2006

(54) Use of the sodium iodine symporter to effect uptake of iodine

(75) Inventors: Frederick E. Domann JR., Iowa City, IA (US); Andrew W. Gaut, Coralville, IA (US); Douglas K. Trask, Coralville, IA (US); Gang Niu, Mountain View, CA (US); Kimberly J. P. Krager, North Liberty, IA (US)

Correspondence Address: FULBRIGHT & JAWORSKI L.L.P., 600 CONGRESS AVE., SUITE 2400, AUSTIN, TX, 78701, US

(73) Assignee: University of Iowa Research Foundation

(21) Appl. No.: 11/345,600

(22) Filed: Feb. 1, 2006

Related U.S. Application Data
(60) Provisional application No. 60/650,400, filed on Feb. 4, 2005.

(51) Int. Cl. A61K 48/00; A61K 33/36; C12N 15/86; A61K 51/00

(52) U.S. Cl. 424/93.2; 435/456; 424/667; 424/1.11

(57) ABSTRACT

The present invention relates to the use of gene therapy to introduce an iodine symporter into cell to increase iodine uptake therein. This approach has particular utility in the treatment of cancers that incapable of removing iodine. In addition, strategies are provided for reducing the export of sodium from cells to improve the efficacy of the iodine symporter gene therapy.

The present application claims benefit of priority to U.S. Provisional Application Ser. No. 60/650,400, filed Feb. 4, 2005, the entire contents of which are hereby incorporated by reference.

### BACKGROUND OF THE INVENTION

I. Field of the Invention

The present invention relates generally to the fields of molecular biology and gene therapy. More particularly, it concerns the use of NIS gene therapy, in conjunction with other elements, to provide increased uptake of iodine in target cells.

II. Description of Related Art

There is a clear need for new treatment options targeting head and neck cancer. Despite advances in the management of head and neck squamous cell carcinoma (HNSCC), survival rates have failed to improve significantly over the past 30 years. Surgical management remains a mainstay of treatment, but it is associated with considerable mortality, functional impairment, and cosmetic deformity. HNSCC has proven remarkably resistant to chemotherapy. The best agents have response rates of less than 50% (Moyer et al., 2004). External beam radiation therapy (EBRT) remains a key treatment modality of HNSCC, either alone or in combination with surgical resection. However, the success of radiation therapy is diminished by normal tissue toxicity and its high complication rate. As an alternative to EBRT, brachytherapy has been attempted in HNSCC.

Brachytherapy uses surgically implanted “seeds” of radioactive materials to deliver ionizing radiation from within the tumor, theoretically limiting the radiation dose that traverses normal tissue. In practice, however, this modality has been hindered by extremely heterogeneous tumor dose delivery due to the limited number of radioactive sources that can be practically implanted. Thus, areas of the tumor receive suboptimal radiation dose while surrounding areas of normal tissue are exposed to toxic radiation doses (Khuri and Jain, 2004; Branchi et al., 2003).

Given the lack of current therapeutic options for head and neck cancer, a better understanding of the underlying molecular mechanisms, and its relative anatomic accessibility for intratumoral injection, there has been significant interest in gene therapy of HNSCC. Several gene therapy strategies have even been implemented as clinical trials in humans, most notably wild-type p53 (Khuri and Jain, 2004; Nemunaitis and O'Brien, 2002; Edelman and Nemunaitis, 2003). While these trials have met with limited success, their primary disadvantage is the shortcoming that hampers all traditional gene therapy strategies: inefficient gene transfer. Under ideal circumstances, in vivo gene transfer techniques result in only 5-10% transfection efficiency (Lotze and Kost, 2002; Lamont et al., 2001). Thus, a majority of the total tumor cells are unable to express the transfected gene. Even with transfer of the most cytotoxic of genes, only those tumors cells expressing the gene are susceptible.

Several techniques have been attempted to overcome this inefficiency of gene transfer. Targeted strategies aim to increase gene expression only in tumor cells while sparing normal tissues. This can be accomplished either by targeting transfection vehicles to tumor-specific cell surface markers or by ensuring tumor-specific expression through the use of transcriptional targeting. Other gene therapy strategies circumvent low gene transfer efficiency by exploiting the bystander effect, whereby tumor cells transfected with a particular gene are able to induce not only their own cell death, but also the death of surrounding cells. Genetically targeted radiotherapy seeks to combine the theoretical advantages of brachytherapy and gene therapy strategies. Ionizing radiation from radioiodide is concentrated and delivered intratumorally by expression of the exogenously delivered sodium iodide symporter (Na⁺/I⁻Symporter, NIS) gene. Thus, the bystander effect can be exploited and the limitations of traditional gene therapy can be mitigated. Furthermore, since the radiation emanates from countless radioiodide molecules instead of only a few larger sources, the dose heterogeneity associated with brachytherapy can be overcome.

Unfortunately, despite some successes, the use of the NIS gene in cancer gene therapy has not been uniformly successful. A likely issue is the ability of certain cells to rapidly exclude iodine even when it is efficiently imported into the cells in the first place. Thus, strategies are needed to identify those cells that are compatible with NIS-based therapy, and also, to identify ways of rendering cells that are incompatible more susceptible to such treatments.

### SUMMARY OF THE INVENTION

In one aspect of the present invention the inventors disclose a method of rendering a cell susceptible to iodine uptake comprising assessing the ability of said cell to export iodine and introducing into said cell an expression construct encoding an iodine symporter. In non-limiting embodiments, the cell can be a cancer cell. The cancer cell can be a squamous cell carcinoma. In further aspects, the assessing comprises measuring iodide retention in cells as a function of time. The expression construct, in certain embodiments, can be a viral expression construct. The viral expression construct can be, for example, selected from the group consisting of adenovirus, retrovirus, herpesvirus, adeno-associated virus, and vaccinia virus. In other aspects, the expression construct can be a non-viral expression construct. The non-viral expression construct can be, for example, disposed in a lipid vehicle. In other non-limiting embodiments, the iodine symporter can be NIS. In further aspects of the present invention, the method further comprises administering a radioactive iodine isotope to said cell.

Another embodiment of the present invention includes a method of rendering a cell susceptible to iodine uptake and retention comprising introducing into said cell an expression construct encoding an iodine symporter, and inhibiting pendrin function. The method, for example, can further comprise assessing the ability of said cell to export iodine. In certain aspects, assessing comprises measuring iodide retention in cells as a function of time. The cell, in certain non-limiting aspects, can be a cancer cell. The cancer cell can be, for example, a squamous cell carcinoma. In other aspects, the expression construct can be a viral expression construct. The viral expression construct, in non-limiting embodiments, can be selected from the group consisting of adenovirus, retrovirus, herpesvirus, adeno-associated virus, and vaccinia virus. In another embodiment, the expression construct can be a non-viral expression construct. The non-viral expression construct can be, for example, disposed in a lipid vehicle. In a non-limiting embodiment, the iodine symporter can be NIS. The method can additionally comprise administering a radioactive iodine isotope to said cell. In other aspects, inhibiting pendrin function comprises inhibiting pendrin expression or administering to the cell an agent that binds to pendrin, or both. Inhibiting pendrin expression, for example, can comprise inhibiting pendrin transcription or translation, or both.

In still another embodiment of the present invention, there is disclosed a method of rendering a head and neck squamous carcinoma cell susceptible to radio-iodine therapy comprising introducing into said cell an expression construct encoding an iodine symporter.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

The term “about” means, in general, the stated value plus or minus 5%.

The use of the term “or” in the claims is used to mean “and/or” unless explicitly indicated to refer to alternatives only or the alternative are mutually exclusive, although the disclosure supports a definition that refers to only alternatives and “and/or.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Ad-hNIS-transduced HNSCC cell lines accumulate [¹²⁵I] iodide to higher levels than do breast carcinoma cell lines. Cells were incubated with 2 μCi radioiodide for 1 hour. Data are expressed as the mean±SD of three replicates and are representative of at least three separate experiments. The fold increase in radioiodide uptake compared with untransduced cells is indicated for each cell line. [symbol] Ad-hNIS; [symbol] Ad-Bgl II; □ untransduced control; [symbol] empty well.

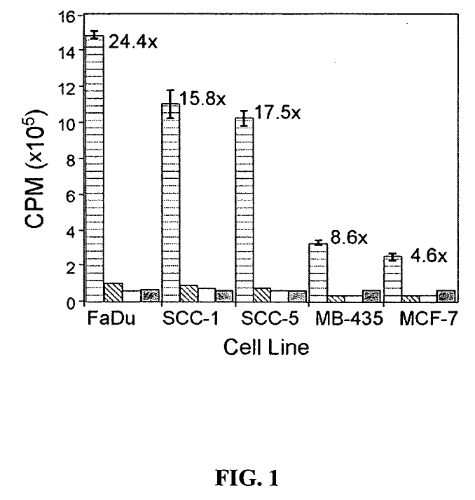

FIG. 2—Perchlorate inhibits NIS-mediated [¹²⁵I] iodide uptake in FaDu cells. Perchlorate (1 mM) was added to the media before the addition of radioiodide. Data are expressed as the mean±SD of three replicates. [symbol] Ad-hNIS; [symbol] Ad-hNIS+1 mM sodium perchlorate; [symbol] Ad-Bgl II; □ untransduced control.

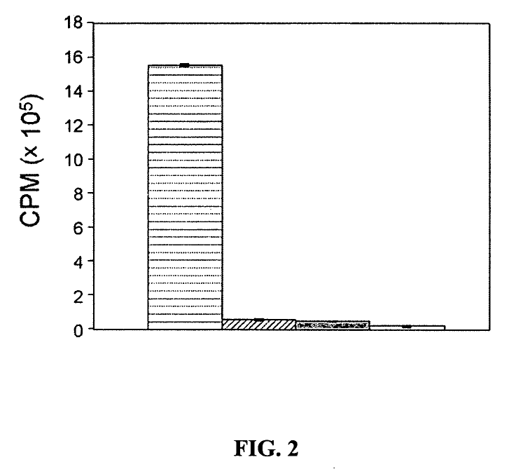

FIG. 3—HNSCC cells exhibit significant retention of [¹²⁵I] iodide. Ad-hNIS-transduced cells were incubated with 2 μCi [¹²⁵I] iodide for 1 hour before washout (time 0). Data are expressed as amount of the radioiodide remaining relative to time 0 and are representative of at least three separate experiments. -•- FaDu; -∘- SCC-1; -▾- SCC-5; -∇- MB-435; -▪- MCF-7.

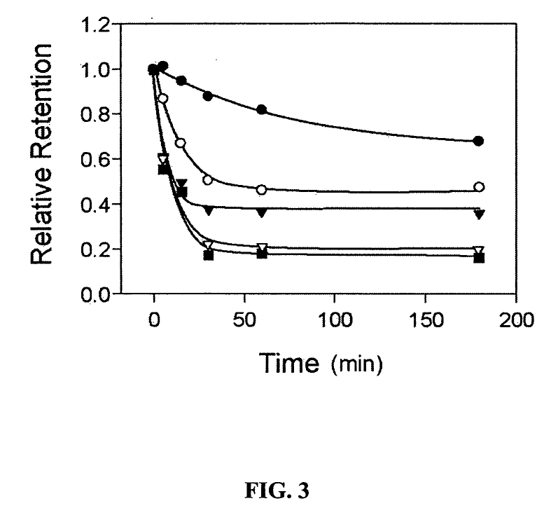

FIG. 4—Ad-hNIS and [¹³¹I] iodide treatment results in a dose-dependent decrease in survival in the HNSCC cell line FaDu. Data are expressed as the mean±SD of three replicates. All pairwise comparisons were statistically significant (p<0.05). [symbol] untransduced control+100 μCi ¹³¹I; [symbol] Ad-Bgl II+100 μCi ¹³¹I; [symbol] Ad-hNIS+100 μCi ¹³¹I; [symbol] Ad-hNIS+200 μCi ¹³¹I; □ Ad-hNIS+300 μCi ¹³¹I.

FIG. 5-Ad-hNIS treatment of FaDu cells followed by systemic delivery of [¹³¹I] iodide results in significant tumor growth inhibition in vivo. Data are expressed as mean F SD of three animals. The difference in average growth rates is statistically significant (p<0.05). □ Ad-Bgl II; • Ad-hNIS.

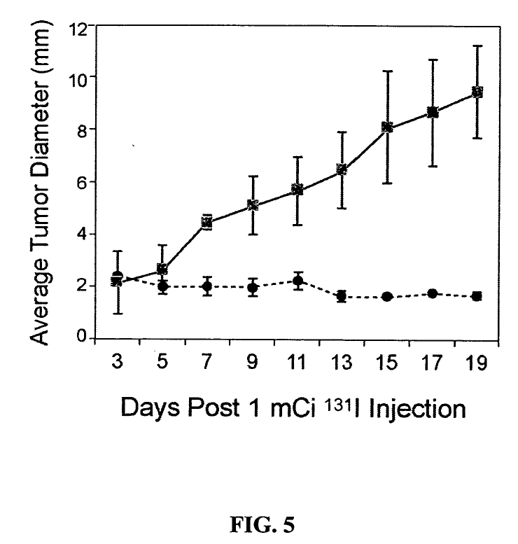

FIG. 6—NIS plus [¹³¹I] therapy caused regression of established human head and neck carcinomas. Tumors in mice were established from hNIS stable expressing FaDu transfectants or parent FaDu cells. When tumors reached an average diameter of 5 mm, 1 mCi [¹³¹I] iodide was administered systemically. Data are expressed as mean±SD of four animals in the FaDu group and eight animals in the NIS-FaDu group. The difference in average growth rates is statistically significant (p<0.05).

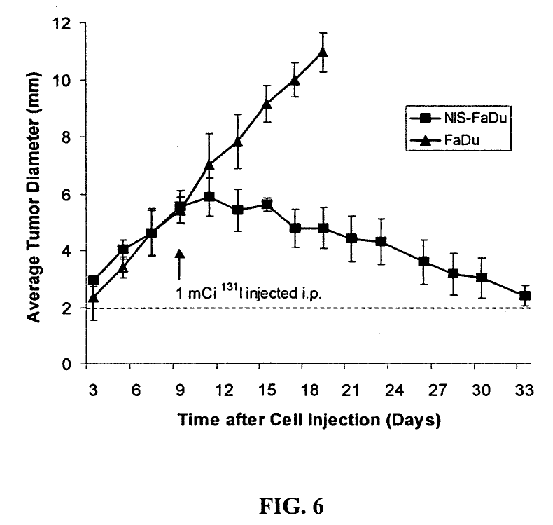

FIG. 7—Intratumoral injection of Ad-NIS followed by systemic ¹³¹I administration causes a significant growth delay in FaDu derived HNSCC tumor xenografts. There was a considerable amount of inter-tumor variability in this experiment compared to the previous experiment. Twenty days after administration of the ¹³¹I there was one tumor that showed a complete response in which the tumor was undetectable; there were four tumors that displayed partial responses, of which two tumors showed significant shrinkage and two tumors displayed growth arrest; and two tumors that were complete non-responders. The heterogeneous pattern of therapeutic response was likely due to individual tumor variability in iodide uptake and retention in the tumors.

FIG. 8—Detection of transgene expression and biodistribution in vivo after intratumoral injection of 1×10⁹ adenovirus particles that express both NIS and GFP genes. Ad-Bgl-II empty virus was used as the control. Low power fields of fluorescence microscopy on frozen sections from tumors injected with Ad-GFP-NIS (left) and Ad-Bgl-II (right) respectively. Sections were counterstained with DAPI. GFP expressing cells are clearly visible by their green fluorescence and are widely distributed throughout the tumor. Bright blue streaks were wrinkles in the tissue sections. Bar=1.0 mm.

FIG. 9—Detection of radioisotope distribution in Ad-NIS infected tumors is relatively homogeneous as determined by cellular level autoradiography of tumor slices. Left panel is a FaDu tumor that was injected with 1×10¹⁰ pfu Ad-NIS. 24 hours later the mouse was administered 1 mCi of iodine-125 (¹²⁵I) by i.p injection. After 6 hours the mouse was euthanized and the tumor was collected, fixed in buffered formalin overnight. Tissue was immersed in tissue freezing medium and snap frozen in liquid nitrogen for further tissue processing by emulsion autoradiography. Right panel is an identically treated FaDu tumor that was injected with Ad-Bgl II empty vector control. The silver grains on the radiographic emulsion illustrate that radioisotope was reasonably well distributed in the Ad-NIS injected tumor, although there is a discernible concentration of silver grains in the lower left hand portion of the upper field. Bar=100 μm.

FIG. 10—Single slice dynamic microSPECT detection of radioisotope distribution and retention in a human HNSCC xenografted tumor. The left tumor was injected with Ad-Bgl-II and the right tumor with Ad-NIS; 48 hours later the mouse was given 5 mCi of ^{99m}Tc as radiotracer and then imaged through time. Each SPECT panel from top left to bottom right is a different time point. In contrast to cells in vitro, tumors show appreciable iodine retention kinetics that are likely responsible for the therapeutic effects observed in vivo. The central necrotic region of the tumor displayed low activity.

FIG. 11—Radioisotope retention in FaDu tumors in vivo is long-lived in contrast to the relatively short retention times observed in vitro. Also note the large variability of uptake (large error bars) in the NIS+ tumors; this may account for the heterogeneity of the therapeutic responses observed in FIG. 7 and accompanying text.

FIG. 12—Pin hole collimator gamma camera scintigraphy of ¹³¹I accumulation in a NIS expressing human tumor xenograft of FaDu carcinoma cells. The tumor volume was recorded as 1.21 cm³, thus yielding a dose of 4.2 Gy of continuous ¹³¹I β-irradiation to the tumor. This dose was sufficient to cause a complete regression of this tumor.

FIG. 13—Gamma camera image of a Ad-NIS injected tumor bearing mouse 2 hours after systemic administration of ¹⁸⁸ReO₄⁻—. The same tissues that have affinity for iodide anion, thyroid, stomach, and NIS-expressing tumor tissue, also display high affinity for ¹¹⁸ReO₄⁻.

FIG. 14—Pendrin expression correlates with iodide retention among a panel of human carcinoma cells. Human carcinoma cells that display long iodide retention times such as FaDu and SkBr3 have low pendrin expression, whereas carcinoma cells that show short retention times such as MB-231 and MB-435 have high pendrin expression.

FIG. 15—Incubation of Ad-NIS infected cells with recombinant LPO during the efflux period caused increased levels of cell-associated iodide after 2 hours of efflux.

FIG. 16—Radioisotope biodistribution in a panel of organs from a mouse that had received intratumoral Ad-NIS followed by systemic ^{99m}TcO₄. These data, presented as % ID (initial dose) per g, revealed a tumor to muscle uptake ratio>2, the target value stated in our RAC approved clinical protocol.

### DESCRIPTION OF THE ILLUSTRATIVE EMBODIMENTS

I. The Present Invention

The present invention relates to the use of an iodine symporter, such as NIS, to increase the uptake of iodine in target cells. In particular, target cells are contacted with an expression construct encoding an NIS gene—the target cells may in one embodiment be cancer cells. Of various target cells, some will retain iodine introduced by NIS function naturally, and thus be amenable to iodine-related therapies. However, other target cells will not, and the use of NIS gene therapy on such cells would not, in and of itself, be worthwhile. Thus, assessing the ability of a cell to retain imported iodine constitutes another aspect of the invention. Finally, in the case of cells which export iodine too efficiently to permit NIS-based therapies, the present invention contemplates strategies by which such iodine export can be abrogated.

The details for practicing the present invention are provided in the following pages.

II. Iodine Symporter

NIS is responsible for the physiologic accumulation of iodide. Localized primarily to the basolateral membrane of thyroid follicular cells, NIS is composed of 13 putative transmembrane-spanning regions (Levy et al., 1998). Iodide is transported into the cell against a concentration gradient by utilizing the inward flux of sodium ions generated by the action of the ATP-dependent Na⁺/K⁺ pump. Animal studies and clinical observations have demonstrated some non-specificity in this iodide transport process. In particular, NIS is also able to concentrate pertechnetate (TcO₄—), astatide (At—), and perrhenate (ReO₄—) (Kotzerke et al., 1998; Lin et al., 2000; Larsen et al., 1998). The transport of these anions is competitively inhibited by the anions perchlorate (ClO₄—) and thiocyanate (SCN—) (Larsen et al., 1998). Clinically, the physiological function of NIS is employed both diagnostically and therapeutically. For example, pertechnetate is frequently used for imaging of the thyroid, and thyroid ablation with 131-Iodide (¹³¹ I) is at least partially responsible for the outstanding 10-year survival rates of metastatic thyroid cancer (Mazzaferri, 2000). Both techniques rely upon the intact function of NIS. Following the isolation and cloning of NIS, studies demonstrated the ability of a number of various non-thyroid cell lines to accumulate iodide after gene transfer with NIS. To date, human glioma, hepatoma, melanoma, breast, lung, cervical, colon, pancreatic and prostate carcinoma cell lines have all been reported to concentrate iodine in response to gene transfer of NIS (Cho et al., 2002; Mandell et al., 1999; Groot-Wassink et al., 2002; Nakamoto et al., 2000; Huang et al., 2001). Total iodide accumulation compared to control cells varied from less than ten to greater than 200-fold in these reports, with maximum uptake occurring within one hour of addition of iodide. Recently, the inventors demonstrated that head and neck squamous cell carcinoma cell lines also accumulate iodide following transfer of the NIS gene (Gaut et al., 2004). Furthermore, HNSCC cell lines demonstrate high levels of both iodide concentration and retention as compared to other cell lines. Since the total radiation dose delivered to a tumor is directly proportional to the amount of radioiodide accumulated and retained within the tumor, this makes HNSCC an ideal candidate for treatment using genetically targeted radiotherapy.

III. Polynucleotides

Certain embodiments of the present invention concern nucleic acids encoding an iodine symporter. In certain aspects, both wild-type and mutant versions of these sequences will be employed. The term “nucleic acid” is well known in the art. A “nucleic acid” as used herein will generally refer to a molecule (i.e., a strand) of DNA, RNA or a derivative or analog thereof, comprising a nucleotide base. A nucleotide base includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between about 8 and about 100 nucleotide bases in length. The term “polynucleotide” refers to at least one molecule of greater than about 100 nucleotide bases in length.

In certain embodiments, a “gene” refers to a nucleic acid that is transcribed. In certain aspects, the gene includes regulatory sequences involved in transcription or message production. In particular embodiments, a gene comprises transcribed sequences that encode for a protein, polypeptide or peptide. As will be understood by those in the art, this functional term “gene” includes genomic sequences, RNA or cDNA sequences or smaller engineered nucleic acid segments, including nucleic acid segments of a non-transcribed part of a gene, including but not limited to the non-transcribed promoter or enhancer regions of a gene. Smaller engineered nucleic acid segments may express, or may be adapted to express proteins, polypeptides, polypeptide domains, peptides, fusion proteins, mutant polypeptides and/or the like.

“Isolated substantially away from other coding sequences” means that the gene of interest forms part of the coding region of the nucleic acid segment, and that the segment does not contain large portions of naturally-occurring coding nucleic acid, such as large chromosomal fragments or other functional genes or cDNA coding regions. Of course, this refers to the nucleic acid as originally isolated, and does not exclude genes or coding regions later added to the nucleic acid by the hand of man.

The accession number for the NIS DNA sequence is NM_{—}000453.

A. Preparation of Nucleic Acids

A nucleic acid may be made by any technique known to one of ordinary skill in the art, such as for example, chemical synthesis, enzymatic production or biological production. Non-limiting examples of a synthetic nucleic acid (e.g., a synthetic oligonucleotide), include a nucleic acid made by in vitro chemical synthesis using phosphotriester, phosphite or phosphoramidite chemistry and solid phase techniques such as described in EP 266 032, incorporated herein by reference, or via deoxynucleoside H-phosphonate intermediates as described by Froehler et al. (1986) and U.S. Pat. No. 5,705,629, each incorporated herein by reference. Various mechanisms of oligonucleotide synthesis may be used, such as those methods disclosed in, U.S. Pat. Nos. 4,659,774; 4,816,571; 5,141,813; 5,264,566; 4,959,463; 5,428,148; 5,554,744; 5,574,146; 5,602,244 each of which are incorporated herein by reference.

A non-limiting example of an enzymatically produced nucleic acid include nucleic acids produced by enzymes in amplification reactions such as PCR™ (see for example, U.S. Pat. Nos. 4,683,202 and 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 2001, incorporated herein by reference).

B. Purification of Nucleic Acids

A nucleic acid may be purified on polyacrylamide gels, cesium chloride centrifugation gradients, column chromatography or by any other means known to one of ordinary skill in the art (see for example, Sambrook et al., 2001, incorporated herein by reference). In certain aspects, the present invention concerns a nucleic acid that is an isolated nucleic acid. As used herein, the term “isolated nucleic acid” refers to a nucleic acid molecule (e.g., an RNA or DNA molecule) that has been isolated free of, or is otherwise free of, bulk of cellular components or in vitro reaction components, and/or the bulk of the total genomic and transcribed nucleic acids of one or more cells. Methods for isolating nucleic acids (e.g., equilibrium density centrifugation, electrophoretic separation, column chromatography) are well known to those of skill in the art.

IV. Expression of Nucleic Acids

In accordance with the present invention, it will be desirable to produce an iodine symporter in a cell. Expression typically requires that appropriate signals be provided in the vectors or expression cassettes, and which include various regulatory elements, such as enhancers/promoters from viral and/or mammalian sources that drive expression of the genes of interest in host cells. Elements designed to optimize messenger RNA stability and translatability in host cells may also be included. Drug selection markers may be incorporated for establishing permanent, stable cell clones.

Viral vectors are selected eukaryotic expression systems. Included are adenoviruses, adeno-associated viruses, retroviruses, herpesviruses, lentivirus and poxviruses including vaccinia viruses and papilloma viruses including SV40. Viral vectors may be replication-defective, conditionally-defective or replication-competent. Also contemplated are non-viral delivery systems, including lipid-based vehicles.

A. Vectors and Expression Constructs

The term “vector” is used to refer to a carrier nucleic acid molecule into which a nucleic acid sequence can be inserted for introduction into a cell where it can be replicated and/or expressed. A nucleic acid sequence can be “exogenous” or “heterologous” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Sambrook et al., 2001 and Ausubel et al., 1996, both incorporated herein by reference).

The term “expression vector” refers to any type of genetic construct comprising a nucleic acid coding for a RNA capable of being transcribed. In some cases, RNA molecules are then translated into a protein, polypeptide, or peptide. Expression vectors can contain a variety of “control sequences,” which refer to nucleic acid sequences necessary for the transcription and possibly translation of an operable linked coding sequence in a particular host cell. In addition to control sequences that govern transcription and translation, vectors and expression vectors may contain nucleic acid sequences that serve other functions as well, as described below.

In order to express an iodine symporter, it is necessary to provide an expression vector. The appropriate nucleic acid can be inserted into an expression vector by standard subcloning techniques. The manipulation of these vectors is well known in the art. Examples of fusion protein expression systems are the glutathione S-transferase system (Pharmacia, Piscataway, N.J.), the maltose binding protein system (NEB, Beverley, Mass.), the FLAG system (IBI, New Haven, Conn.), and the 6×His system (Qiagen, Chatsworth, Calif.).

In yet another embodiment, the expression system used is one driven by the baculovirus polyhedron promoter. The gene encoding the protein can be manipulated by standard techniques in order to facilitate cloning into the baculovirus vector. A preferred baculovirus vector is the pBlueBac vector (Invitrogen, Sorrento, Calif.). The vector carrying the gene of interest is transfected into Spodoptera frugiperda (Sf9) cells by standard protocols, and the cells are cultured and processed to produce the recombinant protein. Mammalian cells exposed to baculoviruses become infected and may express the foreign gene only. This way one can transduce all cells and express the gene in dose dependent manner.

There also are a variety of eukaryotic vectors that provide a suitable vehicle in which recombinant polypeptide can be produced. HSV has been used in tissue culture to express a large number of exogenous genes as well as for high level expression of its endogenous genes. For example, the chicken ovalbumin gene has been expressed from HSV using an α promoter. Herz and Roizman (1983). The lacZ gene also has been expressed under a variety of HSV promoters.

Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. Thus, in certain embodiments, expression includes both transcription of a gene and translation of a RNA into a gene product. In other embodiments, expression only includes transcription of the nucleic acid.

In preferred embodiments, the nucleic acid is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

The particular promoter that is employed to control the expression of a nucleic acid is not believed to be critical, so long as it is capable of expressing the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter.

In various other embodiments, the human cytomegalovirus (CMV) immediate early gene promoter, the SV40 early promoter and the Rous sarcoma virus long terminal repeat can be used to obtain high-level expression of transgenes. The use of other viral or mammalian cellular or bacterial phage promoters which are well-known in the art to achieve expression of a transgene is contemplated as well, provided that the levels of expression are sufficient for a given purpose. Tables 1 and 2 list several elements/promoters which may be employed, in the context of the present invention, to regulate the expression of a transgene. This list is not exhaustive of all the possible elements involved but, merely, to be exemplary thereof.

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Additionally any promoter/enhancer combination (as per the Eukaryotic Promoter Data Base EPDB) could also be used to drive expression of a transgene. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

TABLE 1
PROMOTER

Immunoglobulin Heavy Chain
Immunoglobulin Light Chain
T-Cell Receptor
HLA DQ α and DQ β
β-Interferon
Interleukin-2
Interleukin-2 Receptor
MHC Class II 5
MHC Class II HLA-DRα
β-Actin
Muscle Creatine Kinase
Prealbumin (Transthyretin)
Elastase I
Metallothionein
Collagenase
Albumin Gene
α-Fetoprotein
τ-Globin
β-Globin
c-fos
c-HA-ras
Insulin
Neural Cell Adhesion Molecule (NCAM)
α_{1-Antitrypsin}
H2B (TH2B) Histone
Mouse or Type I Collagen
Glucose-Regulated Proteins (GRP94 and GRP78)
Rat Growth Hormone
Human Serum Amyloid A (SAA)
Troponin I (TN I)
Platelet-Derived Growth Factor
Duchenne Muscular Dystrophy
SV40
Polyoma
Retroviruses
Papilloma Virus
Hepatitis B Virus
Human Immunodeficiency Virus
Cytomegalovirus
Gibbon Ape Leukemia Virus

TABLE 2

| Element | Inducer |
|---|---|
| MT II | Phorbol Ester (TPA) |
| | Heavy metals |
| MMTV (mouse mammary tumor | Glucocorticoids |
| virus) | |
| β-Interferon | Poly(rI)X |
| | Poly(rc) |
| Adenovirus 5 E2 | Ela |
| c-jun | Phorbol Ester (TPA), H₂O₂ |
| Collagenase | Phorbol Ester (TPA) |
| Stromelysin | Phorbol Ester (TPA), IL-1 |
| SV40 | Phorbol Ester (TPA) |
| Murine MX Gene | Interferon, Newcastle Disease Virus |
| GRP78 Gene | A23187 |
| α-2-Macroglobulin | IL-6 |
| Vimentin | Serum |
| MHC Class I Gene H-2kB | Interferon |
| HSP70 | Ela, SV40 Large T Antigen |
| Proliferin | Phorbol Ester-TPA |
| Tumor Necrosis Factor | FMA |
| Thyroid Stimulating Hormone α | Thyroid Hormone |
| Gene | |

One will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal and the bovine growth hormone polyadenylation signal, convenient and known to function well in various target cells. Also contemplated as an element of the expression cassette is a terminator. These elements can serve to enhance message levels and to minimize read through from the cassette into other sequences.

A specific initiation signal also may be required for efficient translation of coding sequences. These signals include the ATG initiation codon and adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements (Bittner et al., 1987).

In various embodiments of the invention, the expression construct may comprise a virus or engineered construct derived from a viral genome. The ability of certain viruses to enter cells via receptor-mediated endocytosis and to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986) and adeno-associated viruses. Retroviruses also are attractive gene transfer vehicles (Nicolas and Rubenstein, 1988; Temin, 1986) as are vaccinia virus (Ridgeway, 1988) and adeno-associated virus (Ridgeway, 1988). Such vectors may be used to (i) transform cell lines in vitro for the purpose of expressing proteins of interest or (ii) to transform cells in vitro or in vivo to provide therapeutic polypeptides in a gene therapy scenario.

B. Viral Vectors

Viral vectors are a kind of expression construct that utilizes viral sequences to introduce nucleic acid and possibly proteins into a cell. The ability of certain viruses to infect cells or enter cells via receptor-mediated endocytosis, and to integrate into host cell genome and express viral genes stably and efficiently have made them attractive candidates for the transfer of foreign nucleic acids into cells (e.g., mammalian cells). Vector components of the present invention may be a viral vector that encode one or more candidate substance or other components such as, for example, an immunomodulator or adjuvant for the candidate substance. Non-limiting examples of virus vectors that may be used to deliver a nucleic acid of the present invention are described below.

1. Adenoviral Vectors

a. Virus Characteristics

Adenovirus is a non-enveloped double-stranded DNA virus. The virion consists of a DNA-protein core within a protein capsid. Virions bind to a specific cellular receptor, are endocytosed, and the genome is extruded from endosomes and transported to the nucleus. The genome is about 36 kB, encoding about 36 genes. In the nucleus, the “immediate early” E1A proteins are expressed initially, and these proteins induce expression of the “delayed early” proteins encoded by the E1B, E2, E3, and E4 transcription units. Virions assemble in the nucleus at about 1 day post infection (p.i.), and after 2-3 days the cell lyses and releases progeny virus. Cell lysis is mediated by the E3 11.6K protein, which has been renamed “adenovirus death protein” (ADP).

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNA's issued from this promoter possess a 5′-tripartite leader (TPL) sequence which makes them preferred mRNA's for translation.

Adenovirus may be any of the 51 different known serotypes or subgroups A-F. Adenovirus type 5 of subgroup C is the human adenovirus about which the most biochemical and genetic information is known, and it has historically been used for most constructions employing adenovirus as a vector. Recombinant adenovirus often is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Viruses used in gene therapy may be either replication-competent or replication-deficient. Generation and propagation of the adenovirus vectors which are replication-deficient depends on a helper cell line, the prototype being 293 cells, prepared by transforming human embryonic kidney cells with Ad5 DNA fragments; this cell line constitutively expresses E1 proteins (Graham et al., 1977). However, helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Racher et al. (1995) have disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Adenovirus growth and manipulation is known to those of skill in the art, and exhibits broad host range in vitro and in vivo. This group of viruses can be obtained in high titers, e.g., 10⁹-10¹³ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Animal studies have suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

b. Engineering

As stated above, Ad vectors are based on recombinant Ad's that are either replication-defective or replication-competent. Typical replication-defective Ad vectors lack the E1A and E1B genes (collectively known as E1) and contain in their place an expression cassette consisting of a promoter and pre-mRNA processing signals which drive expression of a foreign gene. These vectors are unable to replicate because they lack the E1A genes required to induce Ad gene expression and DNA replication. In addition, the E3 genes can be deleted because they are not essential for virus replication in cultured cells. It is recognized in the art that replication-defective Ad vectors have several characteristics that make them suboptimal for use in therapy. For example, production of replication-defective vectors requires that they be grown on a complementing cell line that provides the E1A proteins in trans.

Several groups have also proposed using replication-competent Ad vectors for therapeutic use. Replication-competent vectors retain Ad genes essential for replication, and thus do not require complementing cell lines to replicate. Replication-competent Ad vectors lyse cells as a natural part of the life cycle of the vector. An advantage of replication-competent Ad vectors occurs when the vector is engineered to encode and express a foreign protein. Such vectors would be expected to greatly amplify synthesis of the encoded protein in vivo as the vector replicates. For use as anti-cancer agents, replication-competent viral vectors would theoretically be advantageous in that they would replicate and spread throughout the tumor, not just in the initially infected cells as is the case with replication-defective vectors.

Yet another approach is to create viruses that are conditionally-replication competent. Onyx Pharmaceuticals recently reported on adenovirus-based anti-cancer vectors which are replication-deficient in non-neoplastic cells, but which exhibit a replication phenotype in neoplastic cells lacking functional p53 and/or retinoblastoma (pRB) tumor suppressor proteins (U.S. Pat. No. 5,677,178). This phenotype is reportedly accomplished by using recombinant adenoviruses containing a mutation in the E1B region that renders the encoded E1B-55K protein incapable of binding to p53 and/or a mutation(s) in the E1A region which make the encoded E1A protein (p289R or p243R) incapable of binding to pRB and/or p300 and/or p107. E1B-55K has at least two independent functions: it binds and inactivates the tumor suppressor protein p53, and it is required for efficient transport of Ad mRNA from the nucleus. Because these E1B and E1A viral proteins are involved in forcing cells into S-phase, which is required for replication of adenovirus DNA, and because the p53 and pRB proteins block cell cycle progression, the recombinant adenovirus vectors described by Onyx should replicate in cells defective in p53 and/or pRB, which is the case for many cancer cells, but not in cells with wild-type p53 and/or pRB.

Another replication-competent adenovirus vector has the gene for E1B-55K replaced with the herpes simplex virus thymidine kinase gene (Wilder et al., 1999a). The group that constructed this vector reported that the combination of the vector plus gancyclovir showed a therapeutic effect on a human colon cancer in a nude mouse model (Wilder et al., 1999b). However, this vector lacks the gene for ADP, and accordingly, the vector will lyse cells and spread from cell-to-cell less efficiently than an equivalent vector that expresses ADP.

The present inventor has taken advantage of the differential expression of telomerase in dividing cells to create novel adenovirus vectors which overexpress an adenovirus death protein and which are replication-competent in and, preferably, replication-restricted to cells expressing telomerase. Specific embodiments include disrupting E1A's ability to bind p300 and/or members of the Rb family members. Others include Ad vectors lacking expression of at least one E3 protein selected from the group consisting of 6.7K, gp19K, RIDα (also known as 10.4K); RIDβ (also known as 14.5K) and 14.7K. Because wild-type E3 proteins inhibit immune-mediated inflammation and/or apoptosis of Ad-infected cells, a recombinant adenovirus lacking one or more of these E3 proteins may stimulate infiltration of inflammatory and immune cells into a tumor treated with the adenovirus and that this host immune response will aid in destruction of the tumor as well as tumors that have metastasized. A mutation in the E3 region would impair its wild-type function, making the viral-infected cell susceptible to attack by the host's immune system. These viruses are described in detail in U.S. Pat. No. 6,627,190.

Other adenoviral vectors are described in U.S. Pat. Nos. 5,670,488; 5,747,869; 5,932,210; 5,981,225; 6,069,134; 6,136,594; 6,143,290; 6,210,939; 6,296,845; 6,410,010; and 6,511,184; U.S. Publication No. 2002/0028785.

2. AAV Vectors

The nucleic acid may be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno-associated virus (AAV) is an attractive vector system for use in the methods of the present invention as it has a high frequency of integration and it can infect nondividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. Nos. 5,139,941 and 4,797,368, each incorporated herein by reference.

3. Retroviral Vectors

Retroviruses have promise as therapeutic vectors due to their ability to integrate their genes into the host genome, transferring a large amount of foreign genetic material, infecting a broad spectrum of species and cell types and of being packaged in special cell-lines (Miller, 1992).

In order to construct a retroviral vector, a nucleic acid is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136).

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

4. Other Viral Vectors

Other viral vectors may be employed as vaccine constructs in the present invention. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

5. Delivery Using Modified Viruses

A nucleic acid to be delivered may be housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

6. Non-Viral Delivery

Lipid-based non-viral formulations provide an alternative to adenoviral gene therapies. Although many cell culture studies have documented lipid-based non-viral gene transfer, systemic gene delivery via lipid-based formulations has been limited. A major limitation of non-viral lipid-based gene delivery is the toxicity of the cationic lipids that comprise the non-viral delivery vehicle. The in vivo toxicity of liposomes partially explains the discrepancy between in vitro and in vivo gene transfer results. Another factor contributing to this contradictory data is the difference in liposome stability in the presence and absence of serum proteins. The interaction between liposomes and serum proteins has a dramatic impact on the stability characteristics of liposomes (Yang and Huang, 1997). Cationic liposomes attract and bind negatively charged serum proteins. Liposomes coated by serum proteins are either dissolved or taken up by macrophages leading to their removal from circulation. Current in vivo liposomal delivery methods use aerosolization, subcutaneous, intradermal, intratumoral, or intracranial injection to avoid the toxicity and stability problems associated with cationic lipids in the circulation. The interaction of liposomes and plasma proteins is largely responsible for the disparity between the efficiency of in vitro (Felgner et al., 1987) and in vivo gene transfer (Zhu et al., 1993; Philip et al., 1993; Solodin et al., 1995; Liu et al., 1995; Thierry et al., 1995; Tsukamoto et al., 1995; Aksentijevich et al., 1996).

Recent advances in liposome formulations have improved the efficiency of gene transfer in vivo (Templeton et al. 1997; WO 98/07408, incorporated herein by reference). A novel liposomal formulation composed of an equimolar ratio of 1,2-bis(oleoyloxy)-3-(trimethyl ammonio)propane (DOTAP) and cholesterol significantly enhances systemic in vivo gene transfer, approximately 150-fold. The DOTAP:cholesterol lipid formulation is said to form a unique structure termed a “sandwich liposome.” This formulation is reported to “sandwich” DNA between an invaginated bilayer or “vase” structure. Beneficial characteristics of these liposomes include a positive to negative charge or p, colloidal stabilization by cholesterol, two-dimensional DNA packing and increased serum stability.

The production of lipid formulations often is accomplished by sonication or serial extrusion of liposomal mixtures after (I) reverse phase evaporation (II) dehydration-rehydration (III) detergent dialysis and (IV) thin film hydration. Once manufactured, lipid structures can be used to encapsulate compounds that are toxic (chemotherapeutics) or labile (nucleic acids) when in circulation. Liposomal encapsulation has resulted in a lower toxicity and a longer serum half-life for such compounds (Gabizon et al., 1990). Numerous disease treatments are using lipid based gene transfer strategies to enhance conventional or establish novel therapies, in particular therapies for treating hyperproliferative diseases.

Liposomes are vesicular structures characterized by a lipid bilayer and an inner aqueous medium. Multilamellar liposomes have multiple lipid layers separated by aqueous medium. They form spontaneously when lipids are suspended in an excess of aqueous solution. The lipid components undergo self-rearrangement before the formation of structures that entrap water and dissolved solutes between the lipid bilayers (Ghosh and Bachhawat, 1991). Lipophilic molecules or molecules with lipophilic regions may also dissolve in or associate with the lipid bilayer.

The liposomes are capable of carrying biologically active nucleic acids, such that the nucleic acids are completely sequestered. The liposome may contain one or more nucleic acids and is administered to a mammalian host to efficiently deliver its contents to a target cell. The liposomes may comprise DOTAP and cholesterol or a cholesterol derivative. In certain embodiments, the ratio of DOTAP to cholesterol, cholesterol derivative or cholesterol mixture is about 10:1 to about 1:10, about 9:1 to about 1:9, about 8:1 to about 1:8, about 7:1 to about 1:7, about 6:1 to about 1:6, about 5:1 to about 1:5, about 4:1 to about 1:4, about 3:1 to 1:3, more preferably 2:1 to 1:2, and most preferably 1:1. In further preferred embodiments, the DOTAP and/or cholesterol concentrations are about 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, or 30 mM. The DOTAP and/or Cholesterol concentration can be between about 1 mM to about 20 mM, 1 mM to about 18 mM, 1 mM to about 16 mM, about 1 mM to about 14 mM, about 1 mM to about 12 mM, about 1 mM to about 10 mM, 1 to 8 mM, more preferably 2 to 7 mM, still more preferably 3 to 6 mM and most preferably 4 to 5 mM. Cholesterol derivatives may be readily substituted for the cholesterol or mixed with the cholesterol in the present invention. Many cholesterol derivatives are known to the skilled artisan. Examples include but are not limited to cholesterol acetate and cholesterol oleate. A cholesterol mixture refers to a composition that contains at least one cholesterol or cholesterol derivative.

The formulation may also be extruded using a membrane or filter, and this may be performed multiple times. Such techniques are well-known to those of skill in the art, for example in Martin (1990). Extrusion may be performed to homogenize the formulation or limit its size. A contemplated method for preparing liposomes in certain embodiments is heating, sonicating, and sequential extrusion of the lipids through filters of decreasing pore size, thereby resulting in the formation of small, stable liposome structures. This preparation produces liposomal complexes or liposomes only of appropriate and uniform size, which are structurally stable and produce maximal activity.

For example, it is contemplated in certain embodiments of the present invention that DOTAP:Cholesterol liposomes are prepared by the methods of Templeton et al. (1997; incorporated herein by reference). Thus, in one embodiment, DOTAP (cationic lipid) is mixed with cholesterol (neutral lipid) at equimolar concentrations. This mixture of powdered lipids is then dissolved with chloroform, the solution dried to a thin film and the film hydrated in water containing 5% dextrose (w/v) to give a final concentration of 20 mM DOTAP and 20 mM cholesterol. The hydrated lipid film is rotated in a 50° C. water bath for 45 minutes, then at 35° C. for an additional 10 minutes and left standing at room temperature overnight. The following day the mixture is sonicated for 5 minutes at 50° C. The sonicated mixture is transferred to a tube and heated for 10 minutes at 50° C. This mixture is sequentially extruded through syringe filters of decreasing pore size (1 μm, 0.45 μm, 0.2 μm, 0.1 μm).

It also is contemplated that other liposome formulations and methods of preparation may be combined to impart desired DOTAP:Cholesterol liposome characteristics. Alternate methods of preparing lipid-based formulations for nucleic acid delivery are described by Saravolac et al. (WO 99/18933). Detailed are methods in which lipids compositions are formulated specifically to encapsulate nucleic acids. In another liposome formulation, an amphipathic vehicle called a solvent dilution microcarrier (SDMC) enables integration of particular molecules into the bi-layer of the lipid vehicle (U.S. Pat. No. 5,879,703). The SDMCs can be used to deliver lipopolysaccharides, polypeptides, nucleic acids and the like. Of course, any other methods of liposome preparation can be used by the skilled artisan to obtain a desired liposome formulation in the present invention.

C. Vector Delivery and Cell Transformation

Suitable methods for nucleic acid delivery for transformation of an organelle, a cell, a tissue or an organism for use with the current invention are believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by ex vivo transfection (Wilson et al., 1989; Nabel et al., 1989), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (WO 94/09699 and WO 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); and any combination of such methods.

D. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

Other examples of expression systems include STRATAGENE®'S COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

It is contemplated that an iodine symporter may be “overexpressed,” i.e., expressed in increased levels relative to its natural expression in cells. Such overexpression may be assessed by a variety of methods, including radio-labeling and/or protein purification. However, simple and direct methods are preferred, for example, those involving SDS/PAGE and protein staining or western blotting, followed by quantitative analyses, such as densitometric scanning of the resultant gel or blot. A specific increase in the level of the recombinant protein, polypeptide or peptide in comparison to the level in natural cells is indicative of overexpression, as is a relative abundance of the specific protein, polypeptides or peptides in relation to the other proteins produced by the host cell, e.g., visible on a gel.

In some embodiments, the expressed proteinaceous sequence forms an inclusion body in the host cell, the host cells are lysed, for example, by disruption in a cell homogenizer, washed and/or centrifuged to separate the dense inclusion bodies and cell membranes from the soluble cell components. This centrifugation can be performed under conditions whereby the dense inclusion bodies are selectively enriched by incorporation of sugars, such as sucrose, into the buffer and centrifugation at a selective speed. Inclusion bodies may be solubilized in solutions containing high concentrations of urea (e.g., 8M) or chaotropic agents such as guanidine hydrochloride in the presence of reducing agents, such as β-mercaptoethanol or DTT (dithiothreitol), and refolded into a more desirable conformation, as would be known to one of ordinary skill in the art.

E. Multigene Constructs and IRES

In certain embodiments of the invention, the use of internal ribosome binding sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picanovirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

V. Therapeutic Intervention

Genetically targeted radiotherapy consists of transfer of the NIS gene into a population of cells followed by systemic administration of a cytotoxic radionuclide, typically ¹³¹I. NIS-transfected cells accumulate the radionuclide intracellularly, leading to DNA damage and subsequent cell death. The advantage of genetically-targeted radiotherapy over traditional gene therapy techniques is the former provides opportunity for significant bystander cytotoxicity. Despite the fact that only a fraction of cells in a tumor may express NIS, the radionuclide accumulated by those cells yields ionizing radiation that can cross cell membranes and induce cell death in surrounding cells. In an in vivo tumor, the magnitude of this bystander effect is mediated by three factors: the energetic characteristics of the radionuclide, the amount of radionuclide accumulated in the tumor, and the biochemical and physiological half-life of the radionuclide (Chung, 2002). Thus, maximizing uptake and retention of iodide by NIS is critical to the success of genetically targeted radiotherapy.

Rapid iodide efflux in NIS-transfected cells has been observed in a number of studies. In vitro half-times for iodide efflux are typically less than 10 minutes, though Nakamoto et al. reported a relatively long ˜27 minute efflux half-time in a stably transfected breast carcinoma cell line (Groot-Wassink et al., 2002; Nakamoto et al., 2000; Huang et al., 2001; Min et al., 2002). Though few experiments have been performed, in vivo half-times are somewhat longer, typically on the order of 5-10 hours (Cho et al., 2002; Spitzweg et al., 2001). This may be in part due to the poor vascularity of most tumors, which limits the clearance of radioiodide from the tumor. In an effort to increase iodide retention in cancer cells, two groups have attempted to couple NIS gene transfer with delivery of the thyroperoxidase (TPO) gene. TPO, present in normal thyroid cells, catalyzes the iodination (organification) of tyrosine residues on intracellular proteins. Boland et al. transduced human ovarian adenocarcinoma cell lines with adenoviral constructs expressing NIS and TPO. Despite a significant increase in iodide organification, there was no appreciable change in efflux time (Boland et al., 2002). In contrast, Huang et al. were able to show increased iodide uptake and retention in NIS and TPO co-transfected cells (Huang et al., 2001). It is not clear, however, that organification of iodide is an absolute requirement for effective NIS-mediated radiotherapy. Many thyroid carcinomas, for example, have a reduced ability to organify iodide, yet they are still effectively treated with radioiodide (Mandell et al., 1999). Still, short intracellular retention time is viewed by many as the primary obstacle limiting the feasibility of genetically targeted radiotherapy as a viable treatment for cancer.

The effectiveness of a genetically targeted radiotherapy as a therapeutic strategy has been successfully demonstrated both in vitro and in vivo. Both Mandell et al. and Boland et al. have shown the susceptibility of NIS-transfected cancer cells to in vitro killing by ¹³¹I (Mandell et al., 1999; Boland et al., 2002). Cho et al. demonstrated increased survival of rats bearing tumors from an NIS-expressing glioma cell line following ¹³¹I treatment (Cho et al., 2002). Recently, Spitzweg et al. showed that prostate carcinoma xenografts grown in athymic mice underwent a dramatic 84% reduction in volume after adenoviral-NIS transduction and subsequent ¹³¹I administration (Spitzweg et al., 2001). Preliminary results from the inventor demonstrate similar effects in HNSCC cell lines, both in vitro and in vivo. While most studies of genetically targeted radiotherapy have used ¹³¹I as the radioactive isotope, other pseudohalides, used in place of ¹³¹I, may generate increased cyotoxic effects in tumor cells. Perrhenate [ReO₄—], is taken up avidly by NIS. Using stably transfected cell lines, Van Sande et al. demonstrated two- to three-fold higher in vitro uptake of ReO₄ as compared to iodide (Van Sande et al., 2003). The biophysical properties of ¹⁸⁸ReO₄ also make it well suited for use in genetically targeted radiotherapy. The t1/2 of ¹⁸⁸ReO₄ (16.9 hours) closely approximates the in vivo isotope retention time observed by our group and others. Furthermore, ¹⁸⁸ReO₄ decays with emission of a much higher energy α-particle (2.12 Mev) than ¹³¹I. This higher energy translates to a much larger effective radius (8.84 mm) than that of ¹³¹I (1.66 mm). This larger effective range would prove advantageous in treating larger tumors and would also likely overcome any uneven distribution of radionuclide. Dadachovai et al., using in vivo dosimetry in a breast carcinoma model, demonstrated that a dose 4.5× higher could be delivered using ¹⁸⁸ReO₄ in place of ¹³¹I (Dadachova et al., 2002). Furthermore, Shen et al. showed increased survival of rats bearing NIS-expressing F98 gliomas that were treated with ¹⁸⁸ReO₄ instead of ¹³¹I (Shen et al., 2004).

Thus, the present invention contemplates times periods between NIS therapy and subsequent iodine delivery of 1, 2, 3, 4, 5, 6, 7, 8, 9, 10, 11, 12, 18 or 24 hours, 1, 2, 3, 4, 5, 6, 7, 8, 9, 10, 11, 12, 13 or 14 days, three, four, five, six, seven or eight weeks, one two, three four, five, or six months, and up to one year. The present invention may be utilized in a variety of solid cancers, such as brain cancer, head & neck cancer, esophageal cancer, tracheal cancer, lung cancer, liver cancer stomach cancer, colon cancer, pancreatic cancer, breast cancer, cervical cancer, uterine cancer, bladder cancer, prostate cancer, testicular cancer, skin cancer or rectal cancer. It also may be used against lymphomas or leukemias.

Local, region or systemic delivery of NIS expression constructs and/or iodine to patients is contemplated. It is proposed that this approach will provide clinical benefit, defined broadly as any of the following: reducing primary tumor size, reducing occurrence or size of metastasis, reducing or stopping tumor growth, inhibiting tumor cell division, killing a tumor cell, inducing apoptosis in a tumor cell, reducing or eliminating tumor recurrence.

Patients with unresectable tumors may be treated according to the present invention. As a consequence, the tumor may reduce in size, or the tumor vasculature may change such that the tumor becomes resectable. If so, standard surgical resection may be permitted.

VI. Identifying and Modifying Iodine Exporting Cells

A. Screening of Cell for Iodine Export

In one aspect of the present invention, one will perform assays that evaluate the ability of cells to export iodine. This is accomplished by loading the NIS expressing cells with radioiodine for 1 hour after which the non cell-associated iodine is washed away. The amount of radioiodine associated with the cells is then measured as a function of time using phosphoimage analysis.

B. Strategies for Reducing Iodine Export

Pendrin is a 780 amino acid protein that is encoded by the gene (PDS) mutated in Pendred syndrome. Lower natural pendrin activity has been suggested to be a factor in the increased effectiveness of radioactive iodine therapy. Thus, the present inventors seek to decrease pendrin as a way of increasing the effectiveness of NIS gene therapy. The following are strategies than can be employed to reduce pendrin expression.

1. Antisense Constructs

Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme; see below) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

2. Ribozymes

In certain embodiments of the present invention, the nucleic acid of the pharmaceutical compositions and devices set forth herein is a ribozyme. Although proteins traditionally have been used for catalysis of nucleic acids, another class of macromolecules has emerged as useful in this endeavor. Ribozymes are RNA-protein complexes that cleave nucleic acids in a site-specific fashion. Ribozymes have specific catalytic domains that possess endonuclease activity (Kim and Cook, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Cook et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989; Cook et al., 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990). Recently, it was reported that ribozymes elicited genetic changes in some cells lines to which they were applied; the altered genes included the oncogenes H-ras, c-fos' and genes of HIV. Most of this work involved the modification of a target mRNA, based on a specific mutant codon that is cleaved by a specific ribozyme.

3. RNAi

In certain embodiments of the present invention, the nucleic acid of the pharmaceutical compositions and devices set forth herein is an RNAi. RNA interference (also referred to as “RNA-mediated interference” or RNAi) is a mechanism by which gene expression can be reduced or eliminated. Double-stranded RNA (dsRNA) has been observed to mediate the reduction, which is a multi-step process. dsRNA activates post-transcriptional gene expression surveillance mechanisms that appear to function to defend cells from virus infection and transposon activity (Fire et al., 1998; Grishok et al., 2000; Ketting et al., 1999; Lin and Avery et al., 1999; Montgomery et al., 1998; Sharp and Zamore, 2000; Tabara et al., 1999). Activation of these mechanisms targets mature, dsRNA-complementary mRNA for destruction. RNAi offers major experimental advantages for study of gene function. These advantages include a very high specificity, ease of movement across cell membranes, and prolonged down-regulation of the targeted gene (Fire et al., 1998; Grishok et al., 2000; Ketting et al., 1999; Lin and Avery et al., 1999; Montgomery et al., 1998; Sharp et al., 1999; Sharp and Zamore, 2000; Tabara et al., 1999). Moreover, dsRNA has been shown to silence genes in a wide range of systems, including plants, protozoans, fungi, C. elegans, Trypanasoma, Drosophila, and mammals (Grishok et al., 2000; Sharp et al., 1999; Sharp and Zamore, 2000; Elbashir et al., 2001). It is generally accepted that RNAi acts post-transcriptionally, targeting RNA transcripts for degradation. It appears that both nuclear and cytoplasmic RNA can be targeted (Bosher and Labouesse, 2000).

siRNAs must be designed so that they are specific and effective in suppressing the expression of the genes of interest. Methods of selecting the target sequences, i.e., those sequences present in the gene or genes of interest to which the siRNAs will guide the degradative machinery, are directed to avoiding sequences that may interfere with the siRNA's guide function while including sequences that are specific to the gene or genes. Typically, siRNA target sequences of about 21 to 23 nucleotides in length are most effective. This length reflects the lengths of digestion products resulting from the processing of much longer RNAs as described above (Montgomery et al., 1998).

The making of siRNAs has been mainly through direct chemical synthesis; through processing of longer, double-stranded RNAs through exposure to Drosophila embryo lysates; or through an in vitro system derived from S2 cells. Use of cell lysates or in vitro processing may further involve the subsequent isolation of the short, 21-23 nucleotide siRNAs from the lysate, etc., making the process somewhat cumbersome and expensive. Chemical synthesis proceeds by making two single stranded RNA-oligomers followed by the annealing of the two single stranded oligomers into a double-stranded RNA. Methods of chemical synthesis are diverse. Non-limiting examples are provided in U.S. Pat. Nos. 5,889,136, 4,415,723, and 4,458,066, expressly incorporated herein by reference, and in Wincott et al. (1995).

Several further modifications to siRNA sequences have been suggested in order to alter their stability or improve their effectiveness. It is suggested that synthetic complementary 21-mer RNAs having di-nucleotide overhangs (i.e., 19 complementary nucleotides+3′ non-complementary dimers) may provide the greatest level of suppression. These protocols primarily use a sequence of two (2′-deoxy) thymidine nucleotides as the di-nucleotide overhangs. These dinucleotide overhangs are often written as dTdT to distinguish them from the typical nucleotides incorporated into RNA. The literature has indicated that the use of dT overhangs is primarily motivated by the need to reduce the cost of the chemically synthesized RNAs. It is also suggested that the dTdT overhangs might be more stable than UU overhangs, though the data available shows only a slight (<20%) improvement of the dTdT overhang compared to an siRNA with a UU overhang.

Chemically synthesized siRNAs are found to work optimally when they are in cell culture at concentrations of 25-100 nM, but concentrations of about 100 nM have achieved effective suppression of expression in mammalian cells. siRNAs have been most effective in mammalian cell culture at about 100 nM. In several instances, however, lower concentrations of chemically synthesized siRNA have been used (Caplen, et al., 2000; Elbashir et al., 2001).

WO 99/32619 and WO 01/68836 suggest that RNA for use in siRNA may be chemically or enzymatically synthesized. Both of these texts are incorporated herein in their entirety by reference. The enzymatic synthesis contemplated in these references is by a cellular RNA polymerase or a bacteriophage RNA polymerase (e.g., T3, T7, SP6) via the use and production of an expression construct as is known in the art. For example, see U.S. Pat. No. 5,795,715. The contemplated constructs provide templates that produce RNAs that contain nucleotide sequences identical to a portion of the target gene. The length of identical sequences provided by these references is at least 25 bases, and may be as many as 400 or more bases in length. An important aspect of this reference is that the authors contemplate digesting longer dsRNAs to 21-25mer lengths with the endogenous nuclease complex that converts long dsRNAs to siRNAs in vivo. They do not describe or present data for synthesizing and using in vitro transcribed 21-25mer dsRNAs. No distinction is made between the expected properties of chemical or enzymatically synthesized dsRNA in its use in RNA interference.

Similarly, WO 00/44914, incorporated herein by reference, suggests that single strands of RNA can be produced enzymatically or by partial/total organic synthesis. Preferably, single-stranded RNA is enzymatically synthesized from the PCR products of a DNA template, preferably a cloned cDNA template and the RNA product is a complete transcript of the cDNA, which may comprise hundreds of nucleotides. WO 01/36646, incorporated herein by reference, places no limitation upon the manner in which the siRNA is synthesized, providing that the RNA may be synthesized in vitro or in vivo, using manual and/or automated procedures. This reference also provides that in vitro synthesis may be chemical or enzymatic, for example using cloned RNA polymerase (e.g., T3, T7, SP6) for transcription of the endogenous DNA (or cDNA) template, or a mixture of both. Again, no distinction in the desirable properties for use in RNA interference is made between chemically or enzymatically synthesized siRNA.

U.S. Pat. No. 5,795,715 reports the simultaneous transcription of two complementary DNA sequence strands in a single reaction mixture, wherein the two transcripts are immediately hybridized. The templates used are preferably of between 40 and 100 base pairs, and which is equipped at each end with a promoter sequence. The templates are preferably attached to a solid surface. After transcription with RNA polymerase, the resulting dsRNA fragments may be used for detecting and/or assaying nucleic acid target sequences.

4. Single Chain Antibodies

In certain embodiments of the present invention, the nucleic acid of the pharmaceutical compositions and devices set forth herein encodes a single chain antibody. Single-chain antibodies are described in U.S. Pat. Nos. 4,946,778 and 5,888,773, each of which are hereby incorporated by reference.

VII. Other Therapeutic Combinations

In accordance with the present invention, additional therapies may be applied with further benefit to the patients. Such therapies include chemotherapy, surgery, cytokines, toxins, drugs, dietary, or a non-NIS-based gene therapy. Examples are discussed below.

A. Chemotherapy

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing.

B. Subsequent Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

C. Gene Therapy

In another embodiment, the secondary treatment is a non-p53 gene therapy in which a second gene is administered to the subject. Delivery of a vector encoding p53 in conjunction with a second vector encoding one of the following gene products may be utilized. Alternatively, a single vector encoding both genes may be used. A variety of molecules are encompassed within this embodiment, some of which are described below.

1. Tumor Suppressors

p53 currently is recognized as a tumor suppressor gene. High levels of mutant p53 have been found in many cells transformed by chemical carcinogenesis, ultraviolet radiation, and several viruses. The p53 gene is a frequent target of mutational inactivation in a wide variety of human tumors and is already documented to be the most frequently-mutated gene in common human cancers. It is mutated in over 50% of human NSCLC (Hollstein et al., 1991) and in a wide spectrum of other tumors.

The p53 gene encodes a 393-amino acid phosphoprotein that can form complexes with host proteins such as SV40 large-T antigen and adenoviral E1B. The protein is found in normal tissues and cells, but at concentrations which are minute by comparison with transformed cells or tumor tissue. Interestingly, wild-type p53 appears to be important in regulating cell growth and division. Overexpression of wild-type p53 has been shown in some cases to be anti-proliferative in human tumor cell lines. Thus, p53 can act as a negative regulator of cell growth (Weinberg, 1991) and may directly suppress uncontrolled cell growth or indirectly activate genes that suppress this growth. Thus, absence or inactivation of wild-type p53 may contribute to transformation. However, some studies indicate that the presence of mutant p53 may be necessary for full expression of the transforming potential of the gene.

Wild-type p53 is recognized as an important growth regulator in many cell types. Missense mutations are common for the p53 gene and are essential for the transforming ability of the oncogene. A single genetic change prompted by point mutations can create carcinogenic p53, in as much as mutations in p53 are known to abrogate the tumor suppressor capability of wild-type p53. Unlike other oncogenes, however, p53 point mutations are known to occur in at least 30 distinct codons, often creating dominant alleles that produce shifts in cell phenotype without a reduction to homozygosity. Additionally, many of these dominant negative alleles appear to be tolerated in the organism and passed on in the germ line. Various mutant alleles appear to range from minimally dysfunctional to strongly penetrant, dominant negative alleles (Weinberg, 1991).

Casey and colleagues have reported that transfection of DNA encoding wild-type p53 into two human breast cancer cell lines restores growth suppression control in such cells (Casey et al., 1991). A similar effect also has been demonstrated on transfection of wild-type, but not mutant, p53 into human lung cancer cell lines (Takahasi et al., 1992). p53 appears dominant over the mutant gene and will select against proliferation when transfected into cells with the mutant gene. Normal expression of the transfected p53 does not affect the growth of normal or non-malignant cells with endogenous p53. Thus, such constructs might be taken up by normal cells without adverse effects. It is thus proposed that the treatment of p53-associated cancers with wild-type p53 will reduce the number of malignant cells or their growth rate.

The major transitions of the eukaryotic cell cycle are triggered by cyclin-dependent kinases, or CDK's. One CDK, cyclin-dependent kinase 4 (CDK4), regulates progression through the G₁. The activity of this enzyme may be to phosphorylate Rb at late G₁. The activity of CDK4 is controlled by an activating subunit, D-type cyclin, and by an inhibitory subunit p16^{INK4}. The p16^{INK4} has been biochemically characterized as a protein that specifically binds to and inhibits CDK4, and thus may regulate Rb phosphorylation (Serrano et al., 1993; Serrano et al., 1995). Since the p16^{INK4} protein is a CDK4 inhibitor (Serrano, 1993), deletion of this gene may increase the activity of CDK4, resulting in hyperphosphorylation of the Rb protein. p16 also is known to regulate the function of CDK6.

p16^{INK4} belongs to a newly described class of CDK-inhibitory proteins that also includes p15^{INK4B}, p21^{WAF1}, and p27^{K1P1}. The p16^{INK4} gene maps to 9p21, a chromosome region frequently deleted in many tumor types. Homozygous deletions and mutations of the p16^{INK4} gene are frequent in human tumor cell lines. This evidence suggests that the p16^{INK4} gene is a tumor suppressor gene. This interpretation has been challenged, however, by the observation that the frequency of the p16^{INK4} gene alterations is much lower in primary uncultured tumors than in cultured cell lines (Caldas et al., 1994; Cheng et al., 1994; Hussussian et al., 1994; Kamb et al., 1994; Mori et al., 1994; Okamoto et al., 1994; Nobori et al., 1995; Orlow et al., 1994; Arap et al., 1995). However, it was later shown that while the p16 gene was intact in many primary tumors, there were other mechanisms that prevented p16 protein expression in a large percentage of some tumor types. p16 promoter hypermethylation is one of these mechanisms (Merlo et al., 1995; Herman, 1995; Gonzalez-Zulueta, 1995). Restoration of wild-type p16^{INK4} function by transfection with a plasmid expression vector reduced colony formation by some human cancer cell lines (Okamoto, 1994; Arap, 1995). Delivery of p16 with adenovirus vectors inhibits proliferation of some human cancer lines and reduces the growth of human tumor xenografts.

C-CAM is expressed in virtually all epithelial cells (Odin and Obrink, 1987). C-CAM, with an apparent molecular weight of 105 kD, was originally isolated from the plasma membrane of the rat hepatocyte by its reaction with specific antibodies that neutralize cell aggregation (Obrink, 1991). Recent studies indicate that, structurally, C-CAM belongs to the immunoglobulin (Ig) superfamily and its sequence is highly homologous to carcinoembryonic antigen (CEA; designated 3 in Table 1) (Lin and Guidotti, 1989). Using a baculovirus expression system, Cheung et al. (1993) demonstrated that the first Ig domain of C-CAM is critical for cell adhesive activity.

Cell adhesion molecules, or CAM's are known to be involved in a complex network of molecular interactions that regulate organ development and cell differentiation (Edelman, 1985). Recent data indicate that aberrant expression of CAM's maybe involved in the tumorigenesis of several neoplasms; for example, decreased expression of E-cadherin, which is predominantly expressed in epithelial cells, is associated with the progression of several kinds of neoplasms (Edelman and Crossin, 1991; Frixen et al., 1991; Bussemakers et al., 1992; Matsura et al., 1992; Umbas et al., 1992). Also, Giancotti and Ruoslahti (1990) demonstrated that increasing expression of α₅β₁ integrin by gene transfer can reduce tumorigenicity of Chinese hamster ovary cells in vivo. C-CAM now has been shown to suppress tumor growth in vitro and in vivo.

Other tumor suppressors that may be employed according to the present invention include p21, p15, BRCA1, BRCA2, IRF-1, PTEN (MMAC1), RB, APC, DCC, NF-1, NF-2, WT-1, MEN-I, MEN-II, zac1, p73, VHL, FCC, MCC, DBCCR1, DCP4 and p57.

2. Inducers of Cellular Proliferation

The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occurring oncogenic growth factor. In one embodiment of the present invention, it is contemplated that anti-sense mRNA directed to a particular inducer of cellular proliferation is used to prevent expression of the inducer of cellular proliferation.

The proteins FMS, ErbA, ErbB and neu are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

The proteins Jun, Fos and Myc are proteins that directly exert their effects on nuclear functions as transcription factors.

3. Inhibitors of Cellular Proliferation

The tumor suppressor oncogenes function to inhibit excessive cellular proliferation. The inactivation of these genes destroys their inhibitory activity, resulting in unregulated proliferation. The tumor suppressors Rb, p16, MDA-7, PTEN and C-CAM are specifically contemplated.

4. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., BCl_{XL}, Bcl_{W}, Bcl_{S}, Mcl-1, A1, Bfl-1) or counteract Bcl-2 function and promote cell death (e.g., Bax, Bak, Bik, Bim, Bid, Bad, Harakiri).

VIII. Pharmaceutical Compositions

According to the present invention, therapeutic compositions are administered to a subject. The phrases “pharmaceutically” or “pharmacologically acceptable” refer to compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the compositions, vectors or cells of the present invention, its use in therapeutic compositions is contemplated. Supplementary active ingredients also can be incorporated into the compositions.

In various embodiments, agents that might be delivered may be formulated and administered in any pharmacologically acceptable vehicle, such as parenteral, topical, aerosal, liposomal, nasal or ophthalmic preparations. In certain embodiments, formulations may be designed for oral, inhalant or topical administration. In those situations, it would be clear to one of ordinary skill in the art the types of diluents that would be proper for the proposed use of the polypeptides and any secondary agents required.

Administration of compositions according to the present invention will be via any common route so long as the target tissue or surface is available via that route. This includes oral, nasal, buccal, respiratory, rectal, vaginal or topical. Alternatively, administration may be by intratumoral, intralesional, into tumor vasculature, local to a tumor, regional to a tumor, intradermal, subcutaneous, intramuscular, intraperitoneal or intravenous injection (systemic). Such compositions would normally be administered as pharmaceutically acceptable compositions, described supra.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. Routes of administration may be selected from intravenous, intrarterial, intrabuccal, intraperitoneal, intramuscular, subcutaneous, oral, topical, rectal, vaginal, nasal and intraocular.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

In a particular embodiment, liposomal formulations are contemplated. Liposomal encapsulation of pharmaceutical agents prolongs their half-lives when compared to conventional drug delivery systems. Because larger quantities can be protectively packaged, this allows the opportunity for dose-intensity of agents so delivered to cells.

IX. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Materials and Methods

Cell Culture. The cell lines MCF-7, MDA-MB-435, and FaDu were obtained from the American Type Culture Collection (Manassas, Va.). MCF-7 cells were maintained in RPMI 1640 media with 10% fetal bovine serum and 100 U/ml penicillin/streptomycin. All other cell lines were maintained in high glucose Dulbecco's modified Eagle's medium supplemented to a final concentration of 10% fetal bovine serum, 0.1 mM non-essential amino acids, 2 mM glutamine, and 100 U/mL penicillin/streptomycin (Gibco, Grand Island, N.Y.). Cells were cultured at 37° C. in a 5% CO₂ atmosphere. To create stable transfectants, FaDu cells were transfected with 1 μg pcDNA3 plasmid containing full-length human NIS (hNIS) using Superfect transfection reagent (Qiagen, Valencia, Calif.). After 48 hours, cells were trypsinized and replated in complete medium containing 800 μg/mL G418. Stable clones were isolated and characterized for their ability to accumulate radioiodine.

Preparation of a Recombinant NIS-Expressing Adenovirus. A pcDNA3 plasmid containing hNIS was a kind gift from S. Jhiang (The Ohio State University, Columbus, Ohio). The full-length hNIS cDNA was digested from this plasmid and ligated into the pacAd5 CMV K—N pA shuttle vector. This vector was then linearized and recombined with pTG3602 adenoviral vector backbone in BJ5183 bacteria. Recombinant plasmids were chosen, and the presence of hNIS in the E1 deleted region was confirmed by sequencing. These plasmids were then linearized with Pac I and transfected into HEK 293 cells. Adenovirus expressing hNIS (AdhNIS) was harvested after 7 days and purified in a CsCl₂ gradient by ultracentrifugation. Adenovirus was prepared with assistance from the Gene Transfer Vector Core at the University of Iowa. For experiments in which an adenoviral control was needed, Ad-Bgl II, which lacks the CMV promoter and has deleted E1 and E3 regions, was used.

Radioiodide Uptake and Efflux. Cells (2.5×10⁵) were seeded in each well of 12-well plates on day 1. Twelve hours later, 40 multiplicity of infection (MOI; the ratio of infectious viral particles to cells) of adenovirus was added to each well. Twenty-four hours later, 2 μCi of sodium [¹²⁵I] iodide (New England Nuclear, North Billerica, Mass.) was added to each well, and the plates were incubated for 1 hour at 37° C. Our experience, consistent with the published reports, has demonstrated that maximal uptake occurs by 1 hour after addition of radioiodide (unpublished observations). Medium was then removed, and cells were washed once with PBS. Plates were then exposed overnight on a phosphor imaging screen, and signal intensity was determined with a Typhoon 9400 imager (Amersham Biosciences, Buckinghamshire, England). Signal intensity was compared with that of standards of known activity. For experiments demonstrating inhibition of radioiodide accumulation, sodium perchlorate, a competitive inhibitor of NIS uptake, was added to the medium at a final concentration of 1 mM before the addition of radioiodide. To determine the rate of [¹²⁵I] iodide efflux from cells, uptake was performed as previously described.

After allowing 1 hour for uptake to occur, medium was removed from all wells, wells were washed once with PBS, and 1 mL of fresh iodide-free medium was added. At the appropriate time points, medium was removed, and cells were washed once with PBS before determination of [¹²⁵I] iodide activity using the PhosphorImager. Curves were fit to the data using SigmaPlot software (SPSS Science, Chicago, Ill.).

Clonogenic Cell Survival Assay. FaDu cells (2.5×10⁶) were plated in 25-cm² culture flasks on day 1. Twelve hours later, 30 MOI of adenovirus was added to the flasks. After 24 hours, either 100, 200, or 300 μCi of sodium [¹³¹I] iodide was added to the flasks, and the cells were incubated for 1 hour. Cells were then washed three times with PBS, and a diluting excess (30 mL) of fresh iodide-free medium was added to the flasks. Twelve hours later, cells were trypsinized and plated in triplicate at clonogenic density in 60-mm tissue culture dishes. After 20 days, cells were fixed with 70% ethanol and stained with crystal violet. Colonies containing at least 50 cells were counted.

In Vivo Tumor Growth. The effect of Ad-hNIS plus ¹³¹I iodide on tumor growth in vivo was demonstrated two ways. First, to assess the effects of genetically targeted radiotherapy on tumor outgrowth, 2.0×10⁷ FaDu cells were infected in vitro with 30 MOI of either Ad-hNIS or Ad-Bgl II. Twenty-four hours later, cells were trypsinized, washed, and resuspended in PBS. Ad-Bgl II-transduced cells (5×10⁶) were injected subcutaneously into the right flank, and the same number of Ad-hNIS-transduced cells was injected into the left flank of 8-week-old athymic nude female mice (Harlan Sprague Dawley, Indianapolis, Ind.). After allowing 48 hours for engraftment of tumor cells, 1 mCi of [¹³¹I] iodide was injected intraperitoneally into each mouse. Second, to assess the effects of genetically targeted radiotherapy on established tumors, 5×10⁶ stably transfected NIS-expressing FaDu cells or their parental counterparts were injected subcutaneously into both flanks of athymic mice. The tumors were allowed to grow to an average tumor diameter of 5 mm and then 1 mCi of [¹³¹I] iodide was injected intraperitoneally into each mouse. In both experiments, tumor sizes were recorded in three dimensions every 2 days. Average tumor diameters were determined by taking the cube root of tumor width×length×depth. The experiments were terminated when the average tumor diameter exceeded 10 mm. All animal studies were performed in accordance with the Public Health Service policy on Humane Care and Use of Laboratory Animals, the NIH Guide for the Care and Use of Laboratory Animals, and the Animal elfare Act (7 U.S.C. et seq.); the animal use protocol was approved by the Institutional Animal Care and Use Committee (IACUC) of the University of Iowa.

Statistical Analysis. Data are expressed as the mean±standard deviation. For the clonogenic cell survival assay, Tukey's studentized range test was used for multiple pairwise comparisons to control the type I error rate. For the in vivo tumor formation experiment, a paired t test was performed by use of the average slope of each group. Statistical significance was indicated by p<0.05.

### EXAMPLE 2

### Results

Radioiodide Uptake and Efflux. After 1 hour incubation with [¹²⁵I] iodide, Ad-hNIS transduced cell lines demonstrated increased radioiodide accumulation compared with either Ad-Bgl II-transduced or control cells. The HNSCC cell line FaDu exhibited the highest level of [¹²⁵I] iodide uptake, accumulating radioiodide to levels roughly 25 times that of control cells. Interestingly, all of the HNSCC cell lines tested were able to concentrate radioiodide to higher levels than breast carcinoma cell lines did. Perchlorate inhibited iodide accumulation. Thus, the observed iodide uptake was mediated specifically by NIS.

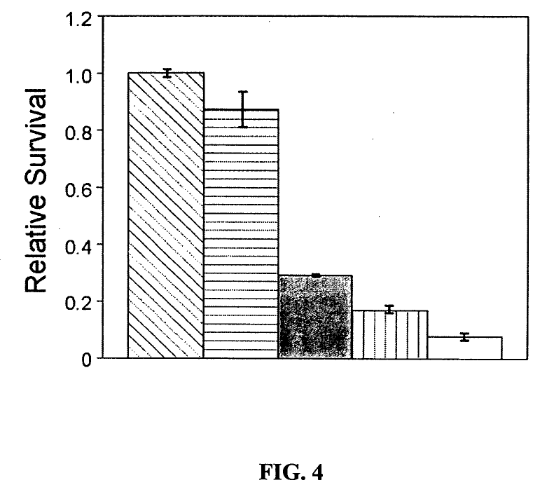

In all tested cell lines, iodide efflux plateaued by 3 hours after [¹²⁵I] iodide washout. However, the terminal levels of iodide retention differed significantly among the different lines. The HNSCC cells lines demonstrated significantly higher levels of retention than did the breast carcinoma cell lines. FaDu, SCC-1, and SCC-5 exhibited 68%, 48%, and 36% of initial [¹²⁵I] iodide levels at 3 hours after washout, respectively. In contrast, MB-435 and MCF-7 breast carcinoma cells retained only 20% and 16% of initial [¹²⁵ I] iodide levels at 3 hours, respectively. Clonogenic Cell Survival Assay (FIG. 4). A clonogenic survival assay was performed to determine the cytotoxic effect of NIS-mediated radioiodide accumulation at varying doses of [¹³¹I] iodide. A statistically significant, dose-dependent decrease in cell survival was apparent in cells transduced with Ad-hNIS. Minimal additional cytotoxicity was observed because of control adenovirus (Ad-Bgl II) treatment plus [¹³¹I] iodide vs radioiodide alone. Thus, the mechanism of cytotoxicity seems to be NIS-mediated cellular accumulation and retention of [¹³¹I] iodide. At the highest dose of [¹³¹I] iodide, Ad-hNIS-treated cells exhibited approximately an 80% absolute decrease in survival over Ad-Bgl II-treated cells.

In Vivo Tumor Growth. To determine the effect of Ad-hNIS-mediated radioiodide accumulation on tumor formation in athymic mice, [¹³¹I] iodide was administered systemically after subcutaneous injection of cells. FaDu cells treated with Ad-hNIS exhibited a dramatic inhibition of tumor growth compared with Ad-Bgl II-transduced controls. By 3 weeks, control tumors had grown by an average of 7.6 mm. In contrast, Ad-hNIS-transduced tumors had decreased by an average of 0.8 mm. Although this experiment demonstrated a profound effect on the ability of the NIS plus [¹³¹I] iodide therapy on preventing the outgrowth of tumors, it did not demonstrate a therapeutic response of this therapy on established tumors. To demonstrate this effect, [¹³¹I] iodide was administered systemically after established tumors that stably expressed the NIS gene had formed in nude mice. After administration of [¹³¹I] iodide, the established tumors showed an almost immediate cessation of growth followed by a period of regression during which eight of eight of the tumors displayed a complete response and shrunk beyond the limit of detection. Sixty days after [¹³¹I] iodide administration, two of the tumors had recurred, whereas the remaining six tumors remained in complete remission. No local adverse effects of radiotherapy were apparent in any of the mice.

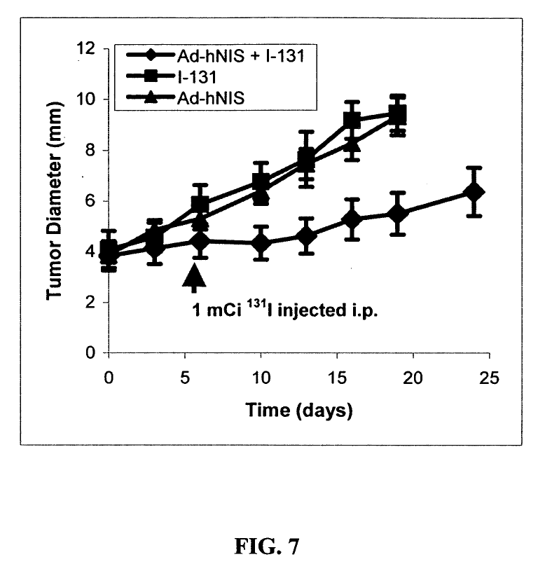

To extend this model system into the most clinically relevant situation, the inventors prepared xenografts of established FaDu tumors that did not express NIS and then used intratumoral injection of high titer adenovirus containing the NIS cDNA. As previously described, NIS expression in the tumors after this treatment could be readily detected and visualized by gamma camera scintigraphy. The major question was whether there would be a significant clinical response of the tumors to ¹³¹I therapy when Ad-NIS was intratumorally injected. Therefore, the inventors designed an experiment in which established FaDu tumors were injected with high titer Ad-NIS along a series of premarked coordinates (feducials) in an attempt to uniformly distribute the gene therapy vector throughout the tumor. Forty-eight hours after administration of the adenovirus the animals were given a systemic administration of 1 mCi ¹³¹I by i.p. injection and tumor dimensions were measured as a function of time. Results of this preliminary experiment are shown in FIG. 7.

From the results of the experiment shown in FIG. 7, it is clear that intratumoral injection of Ad-NIS followed by systemic administration of ¹³¹I provided a heterogeneous response with respect to outcome than the previous experiments with stable NIS-expressing cells. For example, twenty days after administration of the ¹³¹I there was one tumor that showed a complete response in which the tumor was undetectable; there were four tumors that displayed partial responses, of which two tumors showed significant shrinkage and two tumors were growth arrested; and one tumor that was a complete non-responder. These results are probably due to regional variations of NIS expression within the tumor because of the nature of the virus delivery that result in inhomogeneous delivery of radiation dose.

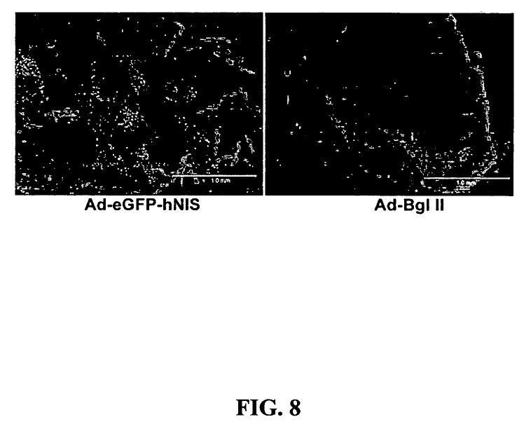

To demonstrate the ability to assess the biodistribution of gene expression within a tumor after intratumoral adenovirus injection, the inventors used a dual expressing virus, Ad-eGFP-NIS that expresses both NIS and the green fluorescent protein. As shown in FIG. 8, green fluorescent cells are visible and distributed throughout the tumor, although they appear to be concentrated in several areas of these sections and we interpret that to be clusters of gene expression along injection needle tracks.

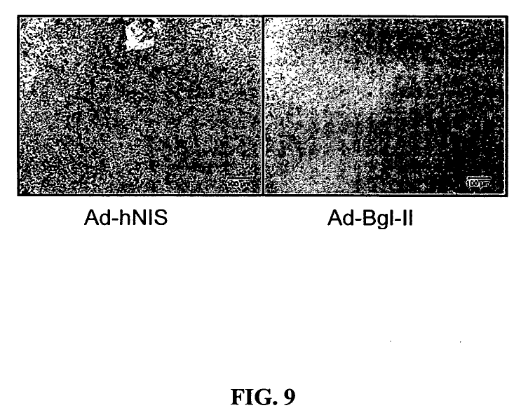

The inventors also performed cellular level autoradiography of the Ad-NIS and empty vector injected tumors using ¹²⁵I as the radiotracer. They wished to see in this preliminary experiment whether they could detect isotope in the tissue by this method, and more importantly what the isotope distribution appeared to be in these tumors. The results of our preliminary cellular level autoradiographic study are shown in FIG. 9, and demonstrate that they can readily detect ¹²⁵I in tumors and there appears to be a relatively homogeneous distribution of radioactivity that can be achieved by Ad-NIS intratumoral injection.

Homogeneity of dose distribution by this therapeutic modality is a very important point and a key difference between this proposed therapy and more traditional seed implants, or brachytherapy. Traditional brachytherapy for head and neck cancer is no longer frequently used because of difficulties controlling dose distribution in the complex anatomy of the head and neck. Nevertheless, brachytherapy has been more successful in cancer of the prostate. A significant drawback to brachytherapy is that inhomogeneous doses from implanted point radiation sources cause a number of complicating sequelae (especially in the head and neck region, e.g., osteoradionecrosis of the jaw, but also likely in other anatomically complex regions as well). The more homogeneous distribution of radioactivity that can be achieved by genetically targeted radiotherapy is an attractive feature of this approach and should generate new enthusiasm in the field of radiation-mediated loco-regional control of human cancer.

The inventors have now extended these preliminary autoradiographic observations using a newly constructed dual reporter construct that expresses not only NIS but also the green fluorescent protein GFP. They have already shown in published work that the two reporters correlate with one another, and it will be technically less demanding to observe the location and distribution of gene expression at the tissue level with the GFP.

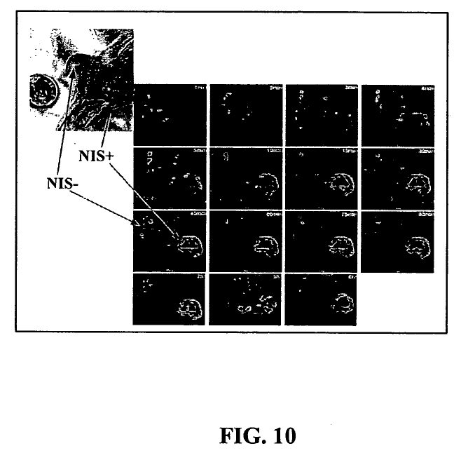

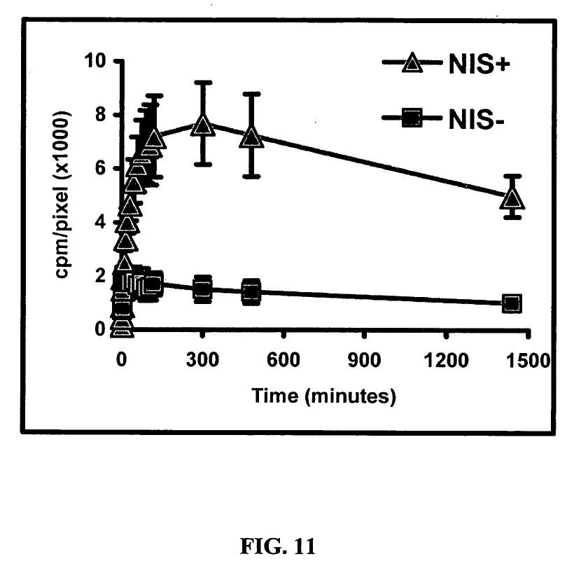

A better method for determining radioisotope distribution in tumors is microSPECT. Quantitative 3-dimensional images of isotope biodistribution can be determined in the tumors of live animals with this method. This is a major advantage since 3-D reconstruction of data from most other analytical methods would be impractical. Moreover, not only uptake but also tumor retention can be measured using dynamic microSPECT imaging. Results of preliminary microSPECT experiments are shown in FIGS. 10 and 11, and reveal the heterogeneity of uptake between different tumors that had been identically treated. The inventors believe that this difference accounts for the heterogeneity of the therapeutic response between tumors, but more importantly, the ability to measure uptake in this manner signifies that one may predict which tumors will respond to therapeutic doses of ¹³¹I.

MicroSPECT biodistribution studies were performed on 4 mice, and the uptake and retention from these microSPECT experiments were quantified and shown in FIG. 11. Unlike the short retention times observed in vitro, in vivo retention is quite long, and this is likely the cause of any observed therapeutic effect. The cause of increased isotope retention in these FaDu tumors in vivo is not yet clear, but it may be related to their relatively poor vascularity (data not shown) and thus poor perfusion of the tumor resulting in iodide “recycling” within the tumor to yield overall greater retention times. Nevertheless, there was a large variation in uptake in the four individual tumors assayed as exhibited by the large error bars on the NIS+ curve in FIG. 11. Among the four tumors investigated, two demonstrated excellent uptake while two others showed only modest uptake. The inventors hypothesize that therapeutic outcome will be predictable based on these types of analyses, and that therapeutic outcome will correlate directly with the area under the curve for iodide uptake and retention.

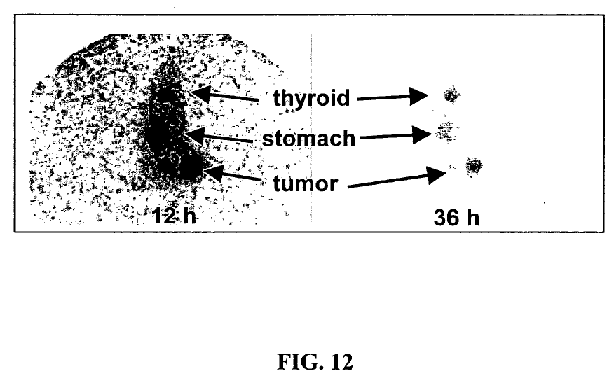

Another important aspect of this approach to deliver radiotherapy to cancer is how much dose can be effectively delivered to the tumor. To begin to address this issue, the inventors have established a method to estimate dosimetry based on gamma camera imaging of ¹³¹I uptake and retention using a pinhole collimator. By using ¹³¹I activity in the tumor as a function of time and knowing the volume of the tumor, the inventors have calculated an estimated radiation dose delivered to a human tumor xenograft. Results of this preliminary experiment are shown in FIG. 12. Data collected from the pinhole collimator gamma camera imaging has enabled us to calculate doses delivered to tumor xenografts and therefore to perform comparative dosimetry between different isotopes such as ¹³¹I and ¹⁸⁸ReO₄—. In the example shown in FIG. 12, the tumor had a volume of 1.21 cm³ and based on the counts acquired by pinhole imaging we were able to determine it had received a dose of 4.2 Gy of continuous ¹³¹I β-irradiation, which was sufficient dose to have caused a remission of this tumor.

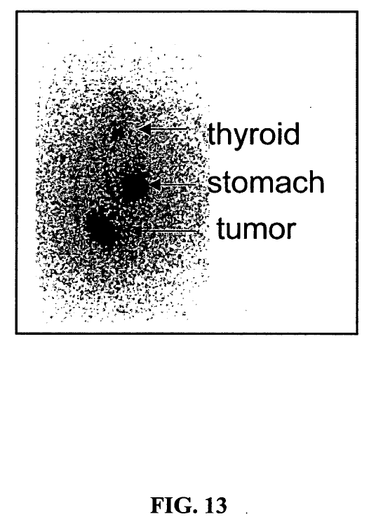

Recently, the inventors have used gamma camera detection to demonstrate that Ad-NIS infected tumors also possess the ability to concentrate another chemical form of cytotoxic radionuclide that is potentially more lethal than ¹³¹I in tumors, ¹⁸⁸Re-perrhennate. Results of this experiment are shown in FIG. 13. As discussed in detail below, the beta particle emitted from ¹⁸⁸Re is more energetic than that of ¹³¹I and can travel a further distance in tissue, thus may be well suited to use in the context of larger tumors that are found in human head and neck cancer.

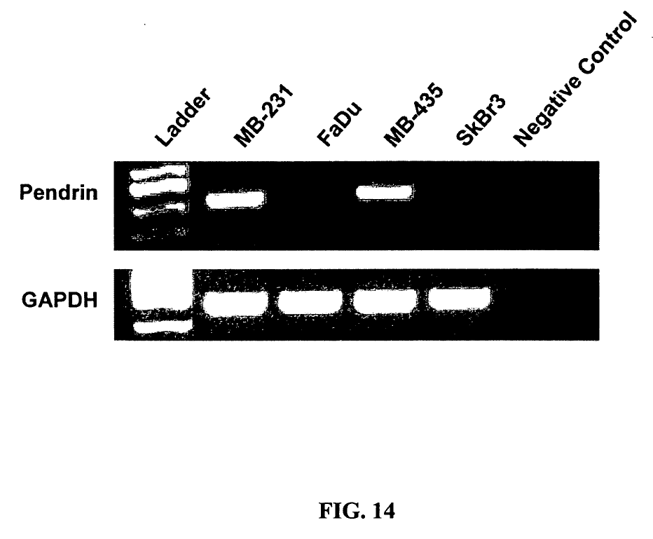

In efforts to understand the molecular mechanisms that govern iodine retention in cells once they have taken it in, the inventors have assessed the expression of a known iodide efflux pump in several of the cell lines we have studied (Scott et al., 1999; Fugazzola et al., 2001). This efflux pump, pendrin, is the protein responsible for iodide efflux into the lumen in thyrocytes and is also the protein affected in the congenital disease Pendred syndrome in which affected individuals present with goiter and deafness. The inventors hypothesized that cell lines that have low iodide retention capacity may have high pendrin expression, whereas cells with high iodide retention characteristics might have low pendrin expression. To begin to test this idea, the inventors performed a pendrin mRNA expression analysis on several of the cell lines in which we already knew the iodide retention characteristics. Results from this preliminary pendrin mRNA expression analysis are shown in FIG. 14. These results suggest that pendrin expression and iodide retention are inversely related and provide a rationale for attempting to modulate pendrin expression as a molecular means to augment cancer cell iodide retention to provide longer radiation exposure times with a goal of increasing the dose that can be delivered to the tumor.

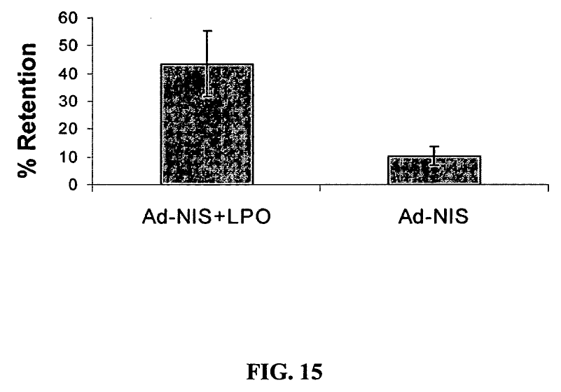

The inventors are also exploring other novel methods to enhance iodine retention in cells and, to this end, they have examined the effect of lactoperoxidase (LPO) on iodide organification and retention in NIS+ cell populations. Results of preliminary experiments indicate that in the presence of LPO, NIS expressing cells exhibit significantly longer retention times of cell-associated iodide than cells without LPO. These data are shown in FIG. 15, and suggest that co-expression of a simple peroxidase could have a significant impact on iodide retention in tumor cells.

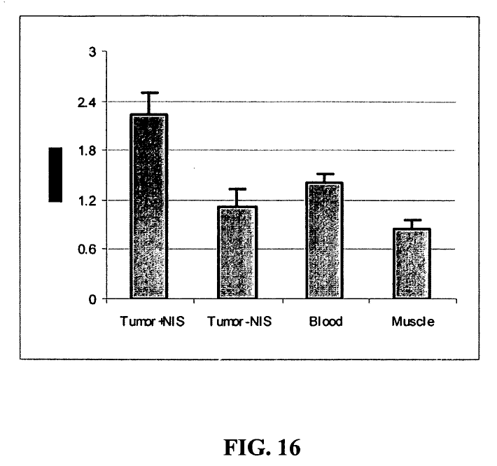

To begin to address the issue of toxicity of genetically targeted radiotherapy, the inventors performed well counting on representative organs from animals that had intratumoral injection of Ad-NIS followed by radioisotope administration. Results of this initial isotope biodistribution study are shown in FIG. 16.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

X. References

The following references, to the extent that they provide exemplary procedural or other details supplementary to those set forth herein, are specifically incorporated herein by reference:
U.S. Pat. No. 4,415,723
U.S. Pat. No. 4,458,066
U.S. Pat. No. 4,659,774
U.S. Pat. No. 4,682,195
U.S. Pat. No. 4,683,202
U.S. Pat. No. 4,797,368
U.S. Pat. No. 4,816,571
U.S. Pat. No. 4,879,236
U.S. Pat. No. 4,946,778
U.S. Pat. No. 4,959,463
U.S. Pat. No. 5,139,941
U.S. Pat. No. 5,141,813
U.S. Pat. No. 5,264,566
U.S. Pat. No. 5,302,523
U.S. Pat. No. 5,322,783
U.S. Pat. No. 5,354,855
U.S. Pat. No. 5,384,253
U.S. Pat. No. 5,428,148
U.S. Pat. No. 5,464,765
U.S. Pat. No. 5,538,877
U.S. Pat. No. 5,538,880
U.S. Pat. No. 5,550,318
U.S. Pat. No. 5,554,744
U.S. Pat. No. 5,563,055
U.S. Pat. No. 5,574,146
U.S. Pat. No. 5,580,859
U.S. Pat. No. 5,589,466
U.S. Pat. No. 5,602,244
U.S. Pat. No. 5,610,042
U.S. Pat. No. 5,645,897
U.S. Pat. No. 5,656,610
U.S. Pat. No. 5,670,488
U.S. Pat. No. 5,677,178
U.S. Pat. No. 5,702,932
U.S. Pat. No. 5,705,629
U.S. Pat. No. 5,736,524
U.S. Pat. No. 5,747,469
U.S. Pat. No. 5,747,869
U.S. Pat. No. 5,780,448
U.S. Pat. No. 5,789,215
U.S. Pat. No. 5,795,715
U.S. Pat. No. 5,871,986
U.S. Pat. No. 5,879,703
U.S. Pat. No. 5,888,773
U.S. Pat. No. 5,889,136
U.S. Pat. No. 5,932,210
U.S. Pat. No. 5,945,100
U.S. Pat. No. 5,981,225
U.S. Pat. No. 5,981,274
U.S. Pat. No. 5,994,136
U.S. Pat. No. 5,994,624
U.S. Pat. No. 6,013,516
U.S. Pat. No. 6,017,524
U.S. Pat. No. 6,069,134
U.S. Pat. No. 6,136,594
U.S. Pat. No. 6,143,290
U.S. Pat. No. 6,210,939
U.S. Pat. No. 6,296,845
U.S. Pat. No. 6,410,010
U.S. Pat. No. 6,511,184
U.S. Pat. No. 6,511,184
U.S. Pat. No. 6,511,847
U.S. Pat. No. 6,627,190
U.S. Appln. 2002/0028785
Aksentijevich et al., Hum. Gene Ther., 7(9):1111-1122, 1996.
Arap et al., Cancer Res., 55(6):1351-1354, 1995.
Ausubel et al., In: Current Protocols in Molecular Biology, John, Wiley & Sons, Inc, New York, 1996.
Baichwal and Sugden, In: Gene Transfer, Kucherlapati (Ed.), NY, Plenum Press, 117-148, 1986.
Bakhshi et al., Cell, 41(3):899-906, 1985.
Bittner et al., Methods in Enzymol, 153:516-544, 1987.
Blomer et al., J. Virol., 71(9):6641-6649, 1997.
Boland et al., Thyroid, 12:19-26, 2002.
Bosher and Labouesse, Nat. Cell. Biol., 2:E31-E36, 2000.
Branchi et al., Minerva Stomatol., 52:411-419, 420-415, 2003.
Bussemakers et al., Cancer Res., 52:2916-2922, 1992.
Caldas et al., Nat. Genet., 8(1):27-32, 1994.
Caplen et al., Gene, 252(1-2):95-105, 2000.
Casey et al., Oncogene, 6(10):1791-1797, 1991.
Chen and Okayama, Mol. Cell Biol., 7(8):2745-2752, 1987.
Cheng et al., Cancer Res., 54(21):5547-5551, 1994.
Cheung et al., Biochem. J, 295:427-435, 1993.
Cho et al., Gene Therapy, 9:1139-1145, 2002.
Chung, J. Nucl. Med., 43:1188-1200, 2002.
Cleary and Sklar, Proc. Natl. Acad. Sci. USA, (21):7439-7443, 1985.
Cleary et al., J. Exp. Med., 164(1):315-320, 1986.
Cook et al., Cell, 27:487-496, 1981.
Cotten et al., Proc. Natl. Acad. Sci. USA, 89(13):6094-6098, 1992.
Couch et al., Am. Rev. Resp. Dis., 88:394-403, 1963.
Coupar et al., Gene, 68:1-10, 1988.
Curiel, Nat. Immun., 13(2-3): 141-164, 1994.
Dadachova et al., Nucl. Med. Biol., 29:13-18, 2002.
Edelman and Crossin, Annu. Rev. Biochem., 60:155-190, 1991
Edelman and Nemunaitis, Curr. Opin. Mol. Ther., 5:611-617, 2003.
Edelman, Annu. Rev. Biochem., 54:135-169, 1985.
Elbashir et al., Nature, 411(6836):494-498, 2001.
EP 266 032
Fechheimer, et al., Proc Natl. Acad. Sci. USA, 84:8463-8467, 1987.
Felgner et al., Proc. Natl. Acad. Sci. USA, 84(21):7413-7417, 1987.
Fire et al., Nature, 391(6669):806-811, 1998.
Forster and Symons, Cell, 49(2):211-220, 1987.
Fraley et al., Proc. Natl. Acad. Sci. USA, 76:3348-3352, 1979.
Friedmann, Science, 244:1275-1281, 1989.
Frixen et al., J. Cell. Biol., 113(1):173-185, 1991.
Froehler et al., Nucleic Acids Res., 14(13):5399-5407, 1986.
Fugazzola et al., Exp. Clin. Endocrinol Diabetes, 109:18-22, 2001.
Gabizon et al., Cancer Res., 50(19):6371-6378, 1990.
Gaut et al., Head Neck, 26:265-271, 2004.
Gerlach et al., Nature (London), 328:802-805, 1987.
Ghosh and Bachhawat, In: Liver Diseases, Targeted Diagnosis and Therapy Using Specific Receptors and Ligands, Wu et al. (Eds.), Marcel Dekker, NY, 87-104, 1991.
Giancotti and Ruoslahti, Cell 60(5):849-859, 1990.
Gomez-Foix et al., J. Biol. Chem., 267:25129-25134, 1992.
Gonzalez-Zulueta et al., Cancer Res., 55(20):4531-4535, 1995.
Gopal, Mol. Cell Biol., 5:1188-1190, 1985.
Graham and Prevec, Biotechnology, 20:363-390, 1992.
Graham and Van Der Eb, Virology, 52:456-467, 1973.
Graham et al, J. General Virology, 36:59-74, 1977.
Grishok et al., Science, 287:2494-2497, 2000.
Groot-Wassink et al., Human Gene Therapy, 13:1723-1735, 2002.
Grunhaus and Horwitz, Seminar in Virology, 3:237-252, 1992.
Harland and Weintraub, J. Cell Biol., 101(3):1094-1099, 1985.
Herman et al., Cancer Res., 55(20):4525-4530, 1995.
Herz and Gerard, Proc. Natl. Acad. Sci. USA, 90:2812-2816, 1993.
Herz and Roizman, Cell, 33(1):145-151, 1983.
Hollstein et al., Science, 253(5015):49-53, 1991.
Horwich et al. J. Virol., 64:642-650, 1990.
Huang et al., Cancer Gene Therapy, 8:612-618, 2001.
Hussussian et al., Nat. Genet., 8(1): 15-21, 1994.
Joyce, Nature, 338:217-244, 1989.
Kaeppler et al., Plant Cell Reports, 9:415-418, 1990.
Kamb et al., Nat. Genet., 8(1):23-26, 1994.
Kaneda et al., Science, 243:375-378, 1989.
Kato et al, J. Biol. Chem., 266:3361-3364, 1991.
Kelleher and Vos, Biotechniques, 17(6):1110-7, 1994.
Kerr et al., Br. J. Cancer, 26(4):239-257, 1972.
Kerr et al., Br. J. Cancer, 26(4):239-257, 1972.
Ketting et al., Cell, 99(2):133-141, 1999.
Khuri and Jain, Semin. Oncol., 31:3-10, 2004.
Kim and Cook, Proc. Natl. Acad. Sci. USA, 84(24):8788-8792, 1987.
Kotzerke et al., Nucl. Med. Commun., 19:795-801, 1998.
Lamont et al., Oncology (Huntingt), 15:303-308, 2001.
Larsen et al., Nucl. Med. Biology, 25:351-357, 1998.
Laughlin et al., J. Virol., 60(2):515-524, 1986.
Le Gal La Salle et al., Science, 259:988-990, 1993.
Lebkowski et al., Mol. Cell. Biol., 8(10):3988-3996, 1988.
Levrero et al., Gene, 101: 195-202, 1991.
Levy et al., J. Bioenerg. Biomem., 30:195-206, 1998.
Lin and Avery, Nature, 402:128-129, 1999.
Lin and Guidotti, J. Biol. Chem., 264:14408-14414, 1989.
Lin et al., Nucl. Med. Biology, 27:83-87, 2000.
Liu et al., Cancer Res., 55(14):3117-3122, 1995.
Lotze and Kost, Cancer Gene Ther., 9:692-699, 2002.
Macejak and Sarnow, Nature, 353:90-94, 1991.
Mandell et al., Cancer Research, 59:661-668, 1999.
Mann et al., Cell, 33:153-159, 1983.
Martin et al., Nature, 345(6277):739-743, 1990.
Matsura et al., Biochim. Biophys. Acta, 1123(3):309-315, 1992.
Mazzaferri, In: The Thyroid: A fundamental and clinical text., Braverman and Utiger (Eds.) 8^{th} Ed., 904-929, Philadelphia: Lippincott, Williams and Wilkins, 2000.
McLaughlin et al., J. Virol., 62(6): 1963-1973, 1988.
Merlo e tal., Nat. Med., 1(7):686-692, 1995.
Michel and Westhof, J. Mol. Biol., 216:585-610, 1990.
Miller et al., Am. J. Clin. Oncol., 15(3):216-221, 1992.
Min et al., Nucl. Med. Biol., 29:537-545, 2002.
Montgomery et al., Proc. Natl. Acad. Sci. USA, 95:15502-15507, 1998.
Mori et al., Cancer Res., 54(13):3396-3397, 1994.
Moyer et al., Curr. Opin. Otolaryngol Head Neck Surg. 12:82-87, 2004.
Muzyczka, Curr. Topics Microbiol. Immunol., 158:97-129, 1992.
Nabel et al., Science, 244(4910):1342-1344, 1989.
Nakamoto et al., J. Nucl. Med., 41:1898-1904, 2000.
Naldini et al., Science, 272(5259):263-267, 1996.
Nemunaitis and O'Brien, Expert Opin. Biol. Ther., 2:311-324, 2002.
Nicolas and Rubenstein, In: Vectors: A survey of molecular cloning vectors and their uses, Rodriguez and Denhardt (Eds.), Stoneham: Butterworth, 494-513, 1988.
Nicolau and Sene, Biochim. Biophys. Acta, 721:185-190, 1982.
Nicolau et al., Methods Enzymol., 149:157-176, 1987.
Nobri et al., Nature (London), 368:753-756, 1995.
Obrink, Bioessays, 13(5):227-234, 1991.
Odin and Obrink, Exp. Cell Res., 171(1):1-15, 1987.
Okamoto et al., Proc. Natl. Acad. Sci. USA, 91(23):11045-11049, 1994.
Orlow et al., Cancer Res, 54(11):2848-2851, 1994.
Paskind et al., Virology, 67:242-248, 1975.
PCT Appln. WO 99/18933
PCT Appln. WO 00/44914
PCT Appln. WO 01/36646
PCT Appln. WO 01/68836
PCT Appln. WO 94/09699
PCT Appln. WO 95/06128
PCT Appln. WO 98/07408
PCT Appln. WO 99/32619
Pelletier and Sonenberg, Nature, 334(6180):320-325, 1988.
Philip et al., J. Biol. Chem., 268(22):16087-16090, 1993.
Potter et al., Proc. Natl. Acad. Sci. USA, 81:7161-7165, 1984.
Racher et al., Biotechnology Techniques, 9:169-174, 1995.
Ragot et al., Nature, 361:647-650, 1993.
Reinhold-Hurek and Shub, Nature, 357:173-176, 1992.
Remington's Pharmaceutical Sciences, 15^{th} ed., pages 1035-1038 and 1570-1580, Mack Publishing Company, Easton, Pa., 1980.
Renan, Radiother. Oncol., 19:197-218, 1990.
Rich et al., Hum. Gene Ther., 4:461-476, 1993.
Ridgeway, In: Vectors: A survey of molecular cloning vectors and their uses, Rodriguez and Denhardt (Eds.), Stoneham:Butterworth, 467-492, 1988.
Rippe et al., Mol. Cell Biol., 10:689-695, 1990.
Rosenfeld et al., Science, 252:431-434, 1991.
Rosenfeld, et al., Cell, 68:143-155, 1992.
Roth et al., Nat Med., 2(9):985-991, 1996.
Roux et al., Proc. Natl. Acad. Sci. USA, 86:9079-9083, 1989.
Sambrook et al., In: Molecular cloning, Cold Spring Harbor Laboratory Press, Cold Spring Harbor, N.Y., 2001.
Sarver et al., Science, 247:1222-1225, 1990.
Scanlon et al., Proc. Natl. Acad. Sci. USA, 88:10591-10595, 1991.
Scott et al., Nat. Genetics, 21:440-443, 1999.
Serrano et al., Nature, 366:704-707, 1993.
Serrano et al., Science, 267(5195):249-252, 1995.
Sharp and Zamore, Science, 287:2431-2433, 2000.
Shen et al., Gene Therapy, 11: 161-169, 2004.
Solodin et al., Biochemistry, 34(41):13537-13544, 1995.
Spitzweg et al., Gene Therapy, 8:1524-1531, 2001.
Stratford-Perricaudet and Perricaudet, In: Human Gene Transfer, Cohen-Haguenauer and Boiron (Eds.), John Libbey Eurotext, France, 51-61, 1991.
Stratford-Perricaudet et al., Hum. Gene. Ther., 1:241-256, 1990.
Tabara et al., Cell, 99(2):123-132, 1999.
Takahasi and Sawasaki, In Vitro Cell Dev. Biol., 28A(6):380-382, 1992.
Temin, In: Gene Transfer, Kucherlapati (Ed.), NY, Plenum Press, 149-188, 1986.
Templeton et al., Nat. Biotechnol., 15(7):647-652, 1997.
Thierry et al., Proc. Natl. Acad. Sci. USA, 92(21):9742-9746, 1995.
Top et al., J. Infect. Dis., 124:155-160, 1971.
Tratschin et al., Mol. Cell. Biol., 4:2072-2081, 1984.
Tsujimoto and Croce, Proc. Natl. Acad. Sci. USA, 83(14):5214-5218, 1986.
Tsujimoto et al., Science, 228(4706):1440-1443, 1985.
Tsukamoto et al., Nat. Genet., 9(3):243-248, 1995.
Tur-Kaspa et al., Mol. Cell Biol., 6:716-718, 1986.
Umbas et al., Cancer Res., 52(18):5104-5109, 1992.
Van Sande et al., Endocrinology, 144:247-252, 2003.
Weinberg, Science, 254(5035):1138-1146, 1991.
Wilder et al., Cancer Res., 59:410-413, 1999a.
Wilder et al., Gene Therapy, 6:57-62, 1999b.
Wilson et al., Science, 244:1344-1346, 1989.
Wincott et al., Nucleic Acids Res., 23(14):2677-2684, 1995.
Wong et al., Gene, 10:87-94, 1980.
Wu and Wu, Biochemistry, 27:887-892, 1988.
Wu and Wu, J. Biol. Chem., 262:4429-4432, 1987.
Yang and Huang, Gene Therapy, 4 (9):950-960, 1997.
Zhu et al., Science, 261(5118):209-211, 1993.
Zufferey et al., Nat. Biotechnol., 15(9):871-875, 1997.

What is claimed is:

1. A method of rendering a cell susceptible to iodine uptake comprising:
(a) assessing the ability of said cell to export iodine; and
(b) introducing into said cell an expression construct encoding an iodine symporter.

2. The method of claim 1, wherein said cell is a cancer cell.

3. The method of claim 2, wherein said cancer cell is a squamous cell carcinoma.

4. The method of claim 1, wherein assessing comprises measuring iodide retention in cells as a function of time.

5. The method of claim 1, wherein said expression construct is a viral expression construct.

6. The method of claim 5, wherein said viral expression construct is selected from the group consisting of adenovirus, retrovirus, herpesvirus, adeno-associated virus, and vaccinia virus.

7. The method of claim 1, wherein said expression construct is a non-viral expression construct.

8. The method of claim 7, wherein said non-viral expression construct is disposed in a lipid vehicle.

9. The method of claim 1, wherein said iodine symporter is NIS.

10. The method of claim 1, further comprising administering a radioactive iodine isotope to said cell.

11. A method of rendering a cell susceptible to iodine uptake and retention comprising:
(a) introducing into said cell an expression construct encoding an iodine symporter; and
(b) inhibiting pendrin function.

12. The method of claim 11, further comprising assessing the ability of said cell to export iodine.

13. The method of claim 12, wherein assessing comprises measuring iodide retention in cells as a function of time.

14. The method of claim 1, wherein said cell is a cancer cell.

15. The method of claim 14, wherein said cancer cell is a squamous cell carcinoma.

16. The method of claim 11, wherein said expression construct is a viral expression construct.

17. The method of claim 16, wherein said viral expression construct is selected from the group consisting of adenovirus, retrovirus, herpesvirus, adeno-associated virus, and vaccinia virus.

18. The method of claim 11, wherein said expression construct is a non-viral expression construct.

19. The method of claim 18, wherein said non-viral expression construct is disposed in a lipid vehicle.

20. The method of claim 11, wherein said iodine symporter is NIS.

21. The method of claim 11, further comprising administering a radioactive iodine isotope to said cell.

22. The method of claim 11, wherein inhibiting pendrin function comprises inhibiting pendrin expression.

23. The method of claim 22, wherein inhibiting pendrin expression comprises inhibiting pendrin transcription.

24. The method of claim 22, wherein inhibiting pendrin expression comprises inhibiting pendrin translation.

25. The method of claim 22, wherein inhibiting pendrin function comprises administering to said cell an agent that binds to pendrin.

26. A method of rendering a head and neck squamous carcinoma cell susceptible to radio-iodine therapy comprising introducing into said cell an expression construct encoding an iodine symporter.
